(19) United States
(12) Patent Application Publication
(10) Pub. No.: US 2025/0062027 A1
(43) Pub. Date: Feb. 20, 2025

(54) MACHINE LEARNING (ML)-BASED SYSTEMS AND METHODS FOR PREDICTING DISEASE

(71) Applicant: AMGEN INC., Thousand Oaks, CA (US)

(72) Inventors: Sze Ling Celine Chui, Pokfulam (HK); Ruibang Luo, Pokfulam (HK); Yekai Zhou, Pokfulam (HK); Ian Chi Kei Wong, Pokfulam (HK)

(21) Appl. No.: 18/807,705

(22) Filed: Aug. 16, 2024

Related U.S. Application Data
(60) Provisional application No. 63/520,554, filed on Aug. 18, 2023.

(51) Int. Cl. G16H 50/20; G16H 10/60; G16H 50/30

(52) U.S. Cl. CPC G16H 50/20; G16H 10/60; G16H 50/30

(57) ABSTRACT

Machine Learning (ML)-based systems and methods are described for predicting cardiovascular disease of users of specific geographic regions. In various aspects, user-specific cardiovascular data of a user may be input into an ML model trained with data of a plurality of cardiovascular risk factors specific to a population of given geographic region. The plurality of cardiovascular risk factors is subdivided into a first training data subset (preselected factors) and a second training data subset (remaining factors). The user-specific cardiovascular data of the user as input into the ML model is data of the user corresponding to the preselected subset of cardiovascular risk factors and the remaining subset of cardiovascular risk factors. The ML model outputs a user-specific cardiovascular prediction of the user. The user-specific cardiovascular prediction comprises a cardiovascular risk score of the user. The cardiovascular prediction is displayed on a graphical user interface (GUI).

### RELATED APPLICATION

This application claims the benefit of U.S. Provisional Application No. 63/520,554 (filed on Aug. 18, 2023), which is incorporated in its entirety by reference herein.

### FIELD OF THE DISCLOSURE

The present disclosure generally relates to artificial intelligence (AI)-based systems and methods, and, more particularly, to machine learning (ML)-based systems and methods for predicting disease (e.g., cardiovascular disease) of users.

### BACKGROUND

Predicting different types of diseases is important for personalized medicine. Cardiovascular disease (CVD) is a leading cause of mortality, especially in developing countries. Cardiovascular diseases (CVD), including coronary heart disease and stroke, are the leading cause of non-communicable deaths globally, with an estimated 18-6 million fatalities recorded in 2019. Cardiovascular diseases can be measured and affect various geographic regions. For example, Cardiovascular diseases are the leading cause of death and disease burden in China, contributing to 3.72 million deaths in 2013 and total hospitalization costs of approximately $14.5 billion (US) in 2016. As a further example, in Hong Kong, heart disease and cerebrovascular diseases were the third and fourth leading cause of deaths in 2021. However, according to a World Health Organization report, 80% of premature heart attacks and strokes are preventable.

### BRIEF SUMMARY

As described herein, ML-based systems and methods are disclosed for predicting disease (e.g., cardiovascular disease) of users. The output of the ML-based systems and methods disclosed herein can be geographically specific, and therefore can account for risk factors, and make predictions for, a given population of that geographic location or region. Further the risk prediction model described herein can be specifically tailored to a specific population for disease prevention and provides dynamic medication treatment with drugs proven to reduce Cardiovascular disease (CVD) risk. In this way, the ML-based systems and methods described herein can provide an important technology to identify and reduce the CVD healthcare burden for a specific geographic region.

In one aspect, a disclosed ML model is trained with data comprising cardiovascular risk factors specific to a specific geographic region in China, which includes one or more geographic regions of China (e.g., Hong Kong). In view of this, the disclosed ML model is referred to herein as the Personalized CARdiovascular DIsease risk Assessment for Chinese (P-CARDIAC) model, which is a specific ML model trained and validated among Chinese population data using Machine-Learning (ML) techniques as described herein. However, it is to be understood that the ML-based systems and methods as described herein may be used with respect to different datasets comprising cardiovascular risk factors specific to additional or different geographic regions having people with additional or different biodiversity.

The ML model (i.e., the P-CARDIAC model), as described herein, can be used to identify patterns in large data sets to enable delivery of healthcare services by facilitating effective patient-provider decision-making. The ML model (e.g., the P-CARDIAC model) can provide early intervention for patients at high risk of recurrent CVD by leveraging a rich data source of electronic health records (EHR). The ML model (i.e., P-CARDIAC) can estimate the 10 years of recurrent CVD risk for high-risk individuals with consideration of an array of risk variables captured in the EHR.

The ML model (i.e., P-CARDIAC), as described herein, can provide predictions of CVD and guidance, treatments, or other output specific to a user, where the guidance, treatments, or other output can comprise information comprising a recommended prescription of one or more drugs or drug classes for treating CVD for a specific user, a user-specific activity for the user (e.g., increased visits to a medical professional), or other such guidance for providing early intervention for a user of the given geographic region (e.g., China) with a high-risk of recurrent CVD.

The performance of the ML model (i.e., P-CARDIAC), as described herein, is more accurate than known techniques involving risk scores for recurrent CVD risk prediction among individuals with established CVD. Such known techniques include TRS-2° P and SMART2. In particular, the ML model (i.e., the P-CARDIAC model) achieves a higher predictive accuracy than TRS-2° P and SMART2 from data cohorts (cohort data between 2004 and 2019) from Hong Kong, a city in Southeast Asia where over 90% of inhabitants are of Chinese ethnicity. In particular, the ML model (i.e., the P-CARDIAC model) has an improved discrimination and calibration with a C-statistic of 0.69 compared with the common risk scores produced by TRS-2° P and SMART2.

In one example embodiment, an ML-based system for predicting cardiovascular disease is disclosed. The ML-based system comprises an ML model stored on a computer memory. The ML model may be trained with data of a plurality of cardiovascular risk factors, which may be specific to a population of a given geographic region. The plurality of cardiovascular risk factors may be subdivided into a first training data subset and a second training data subset prior to training the ML model, wherein the first training data subset may comprise a preselected subset of cardiovascular risk factors, and wherein the second training data subset may comprise a remaining subset of cardiovascular risk factors. The ML-based system may further comprise a set of computing instructions stored on the computer memory and configured to access the ML model. The ML-based system may further comprise a processor communicatively coupled to the computer memory. The processor may be configured to access the set of computing instructions and the ML model. The computing instructions, when executed by the processor, may cause the processor to input user-specific cardiovascular data of the user into the ML model. The user may be a member of the geographic region and the user-specific cardiovascular data of the user as input into the ML model is data of the user corresponding to the preselected subset of cardiovascular risk factors and the remaining subset of cardiovascular risk factors. The computing instructions, when executed by the processor, may further cause the processor to output, by accessing the ML model, a user-specific cardiovascular prediction of the user. The user-specific cardiovascular prediction may comprise a cardiovascular risk score of the user. The computing instructions, when executed by the processor, may further cause the processor to display, by a graphical user interface (GUI), the user-specific cardiovascular prediction.

In an additional example embodiment, an ML-based method for predicting cardiovascular disease is disclosed. The ML-based method comprises training, by one or more processors, an ML model with data of a plurality of cardiovascular risk factors, which may be specific to a population of a given geographic region. The plurality of cardiovascular risk factors may be subdivided into a first training data subset and a second training data subset prior to training the ML model. The first training data subset may comprise a preselected subset of cardiovascular risk factors, and the second training subset may comprise a remaining subset of cardiovascular risk factors. The ML-based method may further comprise inputting, by one or more processors, user-specific cardiovascular data of a user into an ML model stored on a computer memory. The user may comprise a member of the geographic region. The user-specific cardiovascular data of the user as input into the ML model may comprise data of the user corresponding to the preselected subset of cardiovascular risk factors and the remaining subset of cardiovascular risk factors. The ML-based method may further comprise outputting, by one or more processors accessing the ML model, a user-specific cardiovascular prediction of the user. The user-specific cardiovascular prediction may comprise a cardiovascular risk score of the user. The ML-based method may further comprise displaying, by a graphical user interface (GUI), the user-specific cardiovascular prediction.

In a still further embodiment, a tangible, non-transitory computer-readable medium storing computing instructions for predicting cardiovascular disease is disclosed. The computing instructions, when executed by the one or more processors, may cause the one or more processors to train an ML model with data of a plurality of cardiovascular risk factors, which may be specific to a population of a given geographic region. The plurality of cardiovascular risk factors may be subdivided into a first training data subset and a second training data subset prior to training the ML model. The first training data subset may comprise a preselected subset of cardiovascular risk factors, and wherein the second training subset may comprise a remaining subset of cardiovascular risk factors. The computing instructions, when executed by the one or more processors, may further cause the one or more processors to input user-specific cardiovascular data of a user into an ML model stored on a computer memory. The user may comprise a member of the geographic region, and the user-specific cardiovascular data of the user as input into the ML model may comprise data of the user corresponding to the preselected subset of cardiovascular risk factors and the remaining subset of cardiovascular risk factors. The computing instructions, when executed by the one or more processors, may further cause the one or more processors to output, by the ML model, a user-specific cardiovascular prediction of the user, the user-specific cardiovascular prediction comprising a cardiovascular risk score of the user. The computing instructions, when executed by the one or more processors, may further cause the one or more processors to display, by a graphical user interface (GUI), the user-specific cardiovascular prediction.

Additional aspects of the above-mentioned ML-based system, method, and computing instructions stored on the non-transitory computer-readable medium are described in summary as follows.

In some aspects, the ML model is a Cox proportional hazards model.

In additional aspects, a gradient boosting algorithm is implemented or applied to the second training data subset of the remaining subset of cardiovascular risk factors to enhance the Cox proportional hazards model.

In still further aspects, the geographic region defining the plurality of cardiovascular risk factors on which the ML model is trained comprises a plurality subregions or cohorts comprising individuals located within each respective subregion or cohort.

In still further aspects, the preselected subset of cardiovascular risk factors comprises risk factors selected from one or more risk categories defining indications of cardiovascular health.

In still further aspects, the one or more risk categories comprise demographic factors, family history of disease, healthcare utilization, clinical laboratory testing, medication history, disease history, and drug use.

In still further aspects, at least a portion of the preselected subset of cardiovascular risk factors comprise imputed data generated to replace missing values, and wherein the remaining subset of cardiovascular risk factors are not imputed.

In still further aspects, the ML model is further trained with data defining one or more threshold risks, where each threshold risk defines a magnitude of a clinical health benefit to a user of the geographic region.

In still further aspects, a C-statistic for the ML model has a value of at least 0.69.

In still further aspects, the user-specific cardiovascular prediction is a cardiovascular disease (CVD) risk prediction for the user in a 10-year timeframe.

In still further aspects, the ML model is further trained with data of one or more drug classes identified for reducing cardiovascular disease (CVD). In such aspects, the user-specific cardiovascular data of the user as input into the ML model further comprises a selection of one or more of the drug classes. The user-specific cardiovascular prediction of the user may comprise a CVD risk prediction that predicts the user's cardiovascular after using the one or more of the drug classes as selected.

In still further aspects, a GUI is configured to receive the user-specific cardiovascular data of the user. The GUI may be further configured to provide the user-specific cardiovascular data as input to the ML model.

In still further aspects, a GUI provides graphical fields or selections for selecting one or more types of drug classes for selection or generation of a user-specific plan to address the user's cardiovascular health.

In still further aspects, the user-specific cardiovascular prediction comprises a user-specific medical prescription predicted to reduce the user's CVD risk.

In still further aspects, the user-specific cardiovascular prediction causes generation of a user-specific activity predicted to reduce the user's CVD risk.

In accordance with the above, and with the disclosure herein, the present disclosure includes improvements in computer functionality or in improvements to other technologies at least because the claims recite, e.g., the use of a bifurcated and, in many cases, a reduced dataset for training the disclosed ML-model, and using this reduced training dataset to train an ML model without loss of predictive accuracy. In particular, the claims subdividing a plurality of cardiovascular risk factors into a first training subset and a second training data subset prior to training the ML model. The first training subset comprises a preselected subset of cardiovascular risk factors and the second training subset comprises a remaining subset of cardiovascular risk factors. The remaining subset of cardiovascular risk factors may comprise a dataset across hundreds of factors that comprise raw data. In many cases, such raw data includes missing or empty values. However, despite the missing or empty values, disclosed invention allows for training the ML model. That is, the raw data of the second subset of subdivided data need not be updated with additional data or otherwise completed in order to train the ML model to have a high degree of predictive accuracy. Therefore, the present disclosure describes improvements in the functioning of the computer itself or “any other technology or technical field” because the underlying computing device can operate with reduced memory storage (e.g., in need not store complete datasets across all of the risk factors in order to the train or otherwise generate the disclosed ML model). This improves over the prior art at least because existing methodologies require extensive and complete datasets, requiring increase memory storage and processing power in order to successfully train a given model with any degree of accuracy. By contrast, the disclosed ML-based systems and methods for predicting cardiovascular disease can be trained on reduced or otherwise incomplete datasets, while still allowing for accurate predictions. This also increases the speed and efficiency of training the disclosed ML model, as the ML model can be trained and generated with less processing power or resources as compared to known ML training techniques that require larger datasets.

In addition, the present disclosure includes specific features other than what is well-understood, routine, conventional activity in the field, and/or otherwise adds unconventional steps that confine the disclosure to a particular useful application, e.g., machine learning (ML)-based systems and methods for predicting cardiovascular disease of users of specific geographic regions.

Advantages will become more apparent to those of ordinary skill in the art from the following description of the preferred embodiments which have been shown and described by way of illustration. As will be realized, the present embodiments may be capable of other and different embodiments, and their details are capable of modification in various respects. Accordingly, the drawings and description are to be regarded as illustrative in nature and not as restrictive.

### BRIEF DESCRIPTION OF THE DRAWINGS

The Figures described below depict various aspects of the system and methods disclosed therein. It should be understood that each Figure depicts an embodiment of a particular aspect of the disclosed system and methods, and that each of the Figures is intended to accord with a possible embodiment thereof. Further, wherever possible, the following description refers to the reference numerals included in the following Figures, in which features depicted in multiple Figures are designated with consistent reference numerals.

There are shown in the drawings arrangements which are presently discussed, it being understood, however, that the present embodiments are not limited to the precise arrangements and instrumentalities shown, wherein:

FIG. 1 illustrates inclusion and exclusion criteria for generating a dataset with respect to a population of given geographic region for training an ML model, in accordance with various embodiments disclosed herein.

FIG. 2A illustrates a calibration plot for a full ML model (e.g., full P-CARDIAC model), in accordance with various embodiments disclosed herein.

FIG. 2B illustrates a calibration plot for a basic ML model (e.g., basic P-CARDIAC model), in accordance with various embodiments disclosed herein.

FIG. 3 illustrates calibration plots for various models as validated on validation data, in accordance with various embodiments disclosed herein.

FIG. 4 illustrates decision curves of the various models of FIG. 3 with their respective net benefit comparisons, in accordance with various embodiments disclosed herein.

FIG. 5 illustrates an example ML model and an example plurality of cardiovascular risk factors for training the ML model, in accordance with various embodiments disclosed herein.

FIG. 6 illustrates a calibration plot for a full ML model (e.g., full P-CARDIAC model) before recalibration as shown for FIGS. 2A and/or 3, in accordance with various embodiments disclosed herein.

FIG. 7 illustrates an example function of a threshold value defining a risk score as output by the ML model as described for FIG. 5, in accordance with various embodiments disclosed herein.

FIG. 8A illustrates a graphical user interface depicting fields for receiving user-specific data corresponding to a preselected subset of cardiovascular risk factors, in accordance with various embodiments disclosed herein.

FIG. 8B illustrates a graphical user interface depicting output of an ML model of after inputting the values of the preselected subset of cardiovascular risk factors of the GUI of FIG. 8A, in accordance with various embodiments disclosed herein.

FIG. 8C illustrates depicting a graphical user interface depicting fields for receiving user-specific data corresponding to a remaining subset of cardiovascular risk factors, in accordance with various embodiments disclosed herein.

FIG. 8D illustrates a graphical user interface depicting output of the ML model after additionally inputting the values of remaining subset of cardiovascular risk factors, in accordance with various embodiments disclosed herein.

FIG. 8E illustrates a graphical user interface depicting output of the ML model after inputting a selection of one or more of the drug classes, in accordance with various embodiments disclosed herein.

FIG. 8F illustrates a graphical user interface depicting output of the ML model after inputting a second selection of one or more of the drug classes, in accordance with various embodiments disclosed herein.

FIG. 9 illustrates an ML-based method for predicting cardiovascular disease, in accordance with various embodiments disclosed herein.

FIG. 10 illustrates a ML-based system or platform configured to predict cardiovascular disease, in accordance with various embodiments disclosed herein.

The Figures depict preferred embodiments for purposes of illustration only. Alternative embodiments of the systems and methods illustrated herein may be employed without departing from the principles of the invention described herein.

### DETAILED DESCRIPTION

Some research groups advocate the use of risk prediction models on patients to identify those at high risk of Cardiovascular disease (CVD) who are more likely to benefit from preventive strategies. The development and applicability of CVD risk prediction models are highly dependent on the ethnic and socioeconomic factors of the population of interest. Currently, there are several risk scores for recurrent CVD risk prediction among individuals with established CVD, including The Thrombolysis in Myocardial Infarction (TIMI) Risk Score for Secondary Prevention (TRS-2° P) and Secondary Manifestations of ARTerial disease (SMART2) risk score. These risk scores provide an estimated risk of recurrent CVD, and thus help provide early intervention to patients with less resource implications. However, these models are tailored to specific geographic locations having specific populations, whose applicability to other ethnicities is uncertain. Further, there has been limited validation of the influence of ethnicity on the application of existing CVD risk scores, which may be poorly calibrated for target populations for specific geographic regions thereby making such CVD risk scores universally inapplicable. In addition, although treatment options such as lipid-modifying therapies are effective in secondary prevention among those with established CVD, the estimation of treatment effect is often not considered in current risk scores. For the foregoing reasons, there is a need for machine learning (ML)-based systems and methods for predicting cardiovascular disease of users of specific geographic regions.

The present embodiments relate to, inter alia, artificial intelligence systems and methods, and in particular, machine learning ML-based systems and methods for predicting cardiovascular disease. The description herein illustrates data, and ML models trained thereon, which may be specific to a population of given geographic region. However, it is to be understood that different, additional, and/or alternative data may be used, including different, additional, and/or alternative data of other geographic regions in order to achieve the same effects and benefits of the ML-based systems and methods as described herein for predicting cardiovascular disease. An ML model, when trained in accordance with the systems and method disclosed herein, but upon different but similar data of other geographic regions (e.g., a country in Europe), can be configured to provide predictive output for those respective geographic regions. That is, while the examples herein typically refer to China and/or Hong Kong as examples of specific geographic region(s) and/or population(s) it should be understood that additional and/or different data of additional and/or different geographic region(s) and/or population(s) may also be used. By way of non-limiting example, such additional and/or different geographic region(s) and/or population(s) may include and/or comprise geographic or political territories, which may be grouped at various level of geographic-based data granularities, including, for example, any of those of or otherwise associated with Europe, France, and/or Paris; North America, the United States, and/or New York City; Asia, China, and/or Hong Kong; Asia, Japan, and/or Tokyo, and/or other such geographic regions and/or populations, which may be based on continent, country, city, or other geographic or political designations. The ML models and techniques as described herein (referred to as the P-CARDIAC model) can identify patterns in large data sets to enable delivery of healthcare services by facilitating effective patient-provider decision-making. The P-CARDIAC model can be configured to provide early intervention for patients at high risk of recurrent CVD by using as training data, data sources of electronic health records (EHR). The P-CARDIAC model can be trained with multiple years (e.g., 10 years) of recurrent CVD risk for high-risk individuals with consideration of an array of risk variables captured in the EHR. The performance of P-CARDIAC can yield improved results when compared with traditional risk score models (e.g., TRS-2° P, and SMART2 based models).

### Participants

In the examples of this disclosure, patients with established CVD were included in the dataset for training the disclosed ML model if such patients had used any of the public healthcare services provided by the Hong Kong Hospital Authority (HA) since 2004. HA provides government subsidized primary, secondary and tertiary care to all residents, capturing over 70% of all hospitalizations in Hong Kong. The data comprises high validity with a positive predictive value of 85% for myocardial infarction (MI) and 91% for stroke. Three cohorts of Chinese patients were included categorized by their geographical locations; Hong Kong Island cohort as the derivation cohort, whilst the Kowloon and New Territories cohorts were validation cohorts. A total 48,799; 119,672; and 140,533 patients were included in the derivation and validation cohorts, respectively.

### Main Outcomes and Measures

In the examples of this disclosure, the 10-year CVD outcome was a composite of diagnostic or procedure codes for coronary heart disease, ischaemic or hemorrhagic stroke, peripheral artery disease, and revascularization. Incidence of recurrent CVD events was estimated for each cohort with reference to the total person-years of each cohort. Multivariate imputation with chained equations (MICE) and XGBoost were applied for the model development. The comparison with TRS-2° P and SMART2 used the validation cohorts with 1000 bootstrap replicates.

### Results

In the examples of this disclosure, a list of 125 risk variables were used to make predictions on CVD risk, of which, eight classes of medications were considered interactive drug use. Model performance in the derivation cohort showed satisfying discrimination and calibration with a C-statistic of 0.69. Internal validation showed good discrimination and calibration performance with C-statistic over 0.6. P-CARDIAC also showed improved performance compared to TRS-2° P and SMART2 risk scores.

### Conclusions and Relevance

In the examples of this disclosure, compared to other risk scores, an ML model (e.g., the P-CARDIAC model) enables identification of unique patterns of geographically similar users (e.g., Chinese patients) with established CVD. A ML model, such P-CARDIAC or a similar model trained with specific geographic data, can be applied in various settings to prevent recurrent CVD events, thus reducing the related healthcare burden for the given geographic region.

An exemplary list of abbreviations as used herein are provided below.
CVD means Cardiovascular Disease.
P-CARDIAC means Personalized CARdiovascular DIsease risk Assessment for Chinese.
TRS-2° P means Thrombolysis in Myocardial Infarction (TIMI) Risk Score for Secondary Prevention.
SMART2 means Secondary Manifestations of ARTerial disease.
ML means Machine-Learning.
EHR means Electronic Health Records.
HA means Hospital Authority.
ICD-9-CM means Ninth Revision, Clinical Modification.
BNF means British National Formulary.
MICE means Multivariate imputation with chained equations.
CPH means Cox proportional hazards model.
LASSO means Least Absolute Shrinkage and Selection Operator.
CHD means Coronary Heart Disease.
PAD means Peripheral Arterial Disease.
MI means Myocardial Infarction.

### Exemplary Methods

### Study Cohorts

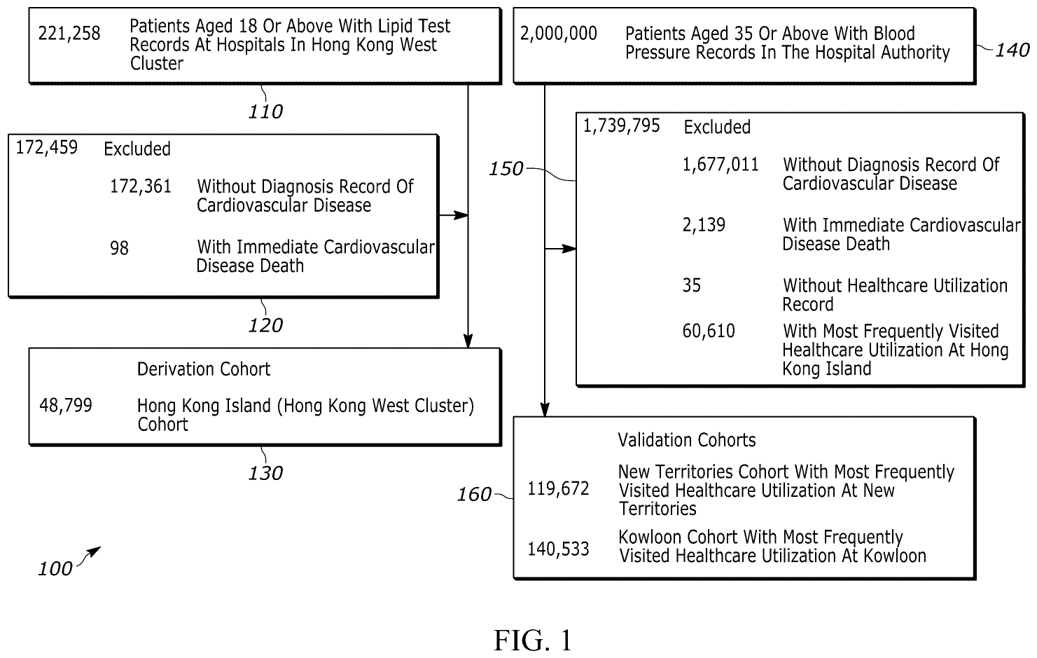

In the examples of this disclosure, three cohorts of patients with established CVD were identified based on geographical location of residence in Hong Kong (Hong Kong West Cluster, Hong Kong Island; Kowloon; New Territories). The Hong Kong Island (Hong Kong West Cluster) cohort was used for model derivation whilst the Kowloon and New Territories cohorts were used for model validation. In various aspects, a geographic region defining a plurality of cardiovascular risk factors on which an ML model is trained comprises a plurality subregions or cohorts (e.g., cohort 130 and/or cohort 160 of FIG. 1) comprising individuals located within each respective subregion or cohort. Patients were included if they had used any of the public healthcare services provided by the Hong Kong Hospital Authority (HA) since 2004 (inclusion and exclusion criteria detailed in FIG. 1 and further herein below). In particular, FIG. 1 illustrates inclusion and exclusion criteria for generating a dataset with respect to a population of given geographic region (e.g., China), where the dataset is used for training an ML model (e.g., ML Model 502 as described herein). FIG. 1 illustrates a non-limiting example for developing or generating data specific to a population of given geographic region (e.g., Hong Kong, China or Paris, France). In various aspects, the developing or generating of data comprises filtering and excluding data of patients in order to define cohorts of data that may, in some aspects, be specific to subregions of the geographic region and/or define discrete patient types. Such data may be used to train an ML model as described herein.

In the example of FIG. 1, data regarding patients aged 18 or above with lipid test records at hospital in a Hong Kong West cluster (110) are considered. Such data is filtered or excluded (120) with respect to patients that fail to have diagnostic record of cardiovascular disease (CVD) or that have died with respect to CVD. By filtering such data, a cohort of data 130 is then established defining data for a cohort of patients regarding Hong Kong Island (Hong Kong West Cluster). Such cohort 130 may comprise a derivation cohort for training an ML model as described herein.

Similarly, as a further example, data regarding patients aged 35 or above with blood pressure records in the Hospital Authority (140) are considered. Such data is filtered or excluded (150) with respect to patients that fail to have diagnostic record of cardiovascular disease (CVD), that have died with respect to CVD, that do not have a utilized healthcare record, or that have been identified as having a most frequently healthcare utilization at Hong Kong Island. By filtering such data, a cohort 160 of data is then established defining data for one or more cohorts of patients regarding New Territories with most frequently visited healthcare utilization in new territories and/or a Kowloon cohort defining data of patients with most frequently visited healthcare utilization in Kowloon. Such cohort 130 may comprise a derivation cohort for training an ML model as described herein.

Additional details of the data as used for cohorts and for training an ML model (as described herein) are described as follows. Such details are described and shown, at least in part, by FIG. 1. A Hong Kong Island (Hong Kong West Cluster) cohort was identified by the Hospital Authority, which included all patients of age 18 or above at the time when they received their lipid test at the hospitals located in Hong Kong West Cluster between 1 Jan. 2004 and 31 Dec. 2019. P-CARDIAC is derived from the Hong Kong Island (Hong Kong West Cluster) cohort.

For the Kowloon and New Territories cohorts, a 2 million patient cohort was retrieved from the Hospital Authority (HA) database. Any patients aged 35-year or above at the time when they had their blood pressure recorded in the Hospital Authority between 1 Jan. 2005 and 31 Dec. 2019. External validation was completed using the Kowloon and Kew Territories cohorts to ensure no overlap with the model derived cohort.

Each patient was categorized as Hong Kong Island (Hong Kong West Cluster), Kowloon, and New Territories based on the region of their most frequently visited healthcare facility within the study period. Cohort entry date was the date of their first diagnosis of CVD in any inpatient and outpatient setting. Patients were censored at the earliest date of the second record of CVD diagnosis, date of registered death, or study end date (31 Dec. 2019). Patients were excluded from the cohort if they had no diagnosis record of CVD, or died on the same day as the first CVD event.

### Outcomes and Risk Variables

In the examples of this disclosure, the outcome is a diagnosis of CVD defined by the International Classification of Diseases, Ninth Revision, Clinical Modification (ICD-9-CM) codes. The outcome comprises a composite of coronary heart disease, ischaemic or hemorrhagic stroke, peripheral artery disease, and revascularization as shown below in Table 1 (showing definitions of cardiovascular disease). The incidence of recurrent CVD events was estimated for each cohort with reference to the total person-years of each cohort.

TABLE 1

| | ICD-9 |
|---|---|
| Diagnosis | |
| Peripheral artery disease | 440, 443.9 |
| Coronary heart disease | 410-414, 429.2, V45.81 |
| Myocardial infarction | 410 |
| Stroke | 430, 431, 432, 433.01, 433.11, 433.21, |
| | 433.31, 433.81, 433.91, 434, |
| | 435, 436, 437.0, 437.1 |
| Procedure | |
| Revascularization | 36.01-36.20 |

A list of an example 125 risk variables including commonly known risk factors such as age, sex, lipid profile, blood pressure, hemoglobin A1c, and blood glucose is shown in Table 2 below. Of the variables in Table 2, 15 variables were identified as preselected risk variables. Such preselected variables were identified or otherwise derived based on clinical evidence, statistically strong correlation and data completeness to predict CVD risk. Example preselected risk variables are indicated in Table 2 with “*” markings. Generally, risk variables may belong to one or more risk categories comprising demographic factors, family history of disease, healthcare utilization, clinical laboratory testing, medication history, disease history, and drug use.

TABLE 2

| Categories (number of | |
|---|---|
| covariates) | Risk variables |
| Demographic factors (2) | age*, sex* |
| Family history of disease (2) | diabetes*, cardiovascular disease |
| Healthcare utilization (3) | accident and emergency visits per year*, inpatient visits per year, |
| | outpatient visits per year |
| Clinical laboratory tests (39) | aspartate transaminase*, alanine aminotransferase*, low-density |
| | lipoprotein cholesterol*, neutrophil*, hemoglobin A1c, creatine |
| | kinase (total), prothrombin time, potassium (serum), estimated |
| | glomerular filtration rate, triglycerides, basophil, arterial partial |
| | pressure of oxygen, albumin, international normalized ratio, |
| | diastolic blood pressure, bicarbonate (serum), glucose (fasting), |
| | erythrocyte sedimentation rate, free thyroxine, troponin I, bilirubin |
| | (total), C-reactive protein, total cholesterol, blood pH, systolic blood |
| | pressure, thyroid stimulating hormone, lymphocyte, creatinine |
| | (serum), platelet, red blood cell, high-density lipoprotein cholesterol, |
| | body mass index, calcium (serum), white blood cell, alkaline |
| | phosphatase, sodium (serum), eosinophil, hemoglobin, monocyte |
| Medication history (prior to | statins*, antihypertensive drugs, antidiabetic drugs, antiplatelet |
| incident CVD event) (27) | drugs, non-steroidal anti-inflammatory drugs, corticosteroids, |
| | proton-pump inhibitors, H2 (histamine type 2)-receptor antagonists, |
| | anticoagulants, nicotine replacement therapy, antiarrhythmic drugs, |
| | antithyroid drugs, oestrogen, psychotropic drugs, cardiac glycosides, |
| | nitrates, thyroid hormones, testosterone, fibrates, niacin, PCSK9 |
| | (Proprotein convertase subtilisin/kexin type 9) inhibitors, cholesterol |
| | absorption inhibitors, Vytorin, bile acid sequestrants, omega-3 fatty |
| | acids, other non-statin lipid-modifying drugs, count of medication |
| Disease history (44) | myocardial infarction*, angina*, revascularization*, atrial |
| | fibrillation*, hypertension*, diabetes*, congestive heart failure, |
| | stroke, thyroid disease, arrhythmia and conduction disorders, |
| | obesity, coronary heart disease, hypothyroidism, cardiac |
| | wall/valve/shunt replacement/repairment, oxygen |
| | therapy/ventilator/intubation, asthma, injury and poisoning, alcohol |
| | user, dyslipidemia, cardiomyopathy, Parkinson's disease, |
| | defibrillator insertion, major organ bleeding, severe mental illness, |
| | dementia, pacemaker implantation, liver disease, chronic obstructive |
| | pulmonary disease, cancer, rheumatoid arthritis, renal disease, |
| | smoker, chronic kidney disease, muscle pain or myopathy or |
| | rhabdomyolysis, dialysis, Creutzfeldt-Jakob disease, cardioversion, |
| | nephrotic syndrome, coronary artery bypass graft, systemic lupus |
| | erythematosus, heart transplantation, peripheral artery disease, |
| | migraine, Down's syndrome |
| Drug use (after incident CVD | antihypertensive drugs, antidiabetic drugs, antiplatelet drugs, statins, |
| event) (8) | fibrates, niacin, PCSK9 inhibitors, cholesterol absorption inhibitors |

Eight classes of medications including lipid-modifying drugs (e.g., fibrates, niacin, cholesterol absorption inhibitors, PCSK9 inhibitors, and statins), antihypertensive, antidiabetic, and antiplatelet drugs (Tables 4 and 5) are considered interactive drug use options (e.g., CVD-related drug use options) for observance of any changes in CVD risk in a given ML model. Diagnoses and procedures are defined by ICD-9-CM codes as shown in Table 3 below (Disease list and related disease codes).

TABLE 3

| Disease/Symptoms | ICD-9-CM code |
|---|---|
| Atrial fibrillation | 427.3 |
| Renal disease | 403.01, 403.11, 403.91, 404.02, 404.03, 404.12, 404.13, 404.92, |
| | 404.93, 580, 582, 583.0-583.7, 585-587, 588.0, 589, 590, 593.0- |
| | 593.2, 593.6, 593.8, 593.9, 599.7, 753.0-753.4, 966.1, V42.0, |
| | V45.1, V56 |
| Chronic kidney disease | 585 |
| Dialysis | 585.9, V56.0, V56.8, 39.95 |
| Congestive heart failure | 428 |
| Diabetes | 250 |
| Down's syndrome | 758.0 |
| Hypertension | 401-405 |
| Arrhythmia and conduction | 426, 427 |
| disorders | |
| Cardiomyopathy | 425 |
| Angina | 413 |
| Coronary artery bypass graft | 414.04, V45.81 |
| Myocardial infarction | 410 |
| Dyslipidaemia | 272 |
| Thyroid disease | 240-244 |
| Liver disease | 570-573 |
| Migraine | 346 |
| Nephrotic syndrome | 581 |
| Rheumatoid arthritis | 446.5, 710.0-710.4, 714.0-714.3, 725 |
| Several mental illnesses | 290-319 |
| Systemic lupus erythematosus | 710.0 |
| Obesity | 278 |
| Dementia | 290, 291, 292.82, 294, 331 |
| Chronic obstructive pulmonary | 490-492, 494, 496 |
| disease | |
| Asthma | 493 |
| Alcohol use | 265.2, 291, 303, 305.0, 357.5, 425.5, |
| | 535.3, 571.0- 571.3, 980, V11.3 |
| Smoker | 305.1, V15.82, V15.83, 649.0 |
| Cancer | 140-209, 230-239 |
| Pacemaker implantation | 37.7, 37.8 |
| Defibrillator insertion | 37.94-37.98 |
| Cardioversion | 99.61 |
| Cardiac wall/valve/shunt | 39.0-39.2 |
| replacement/repairment | |
| Echocardiography | 37.28 |
| Heart transplantation | 37.51 |
| Oxygen | 00.49, 93.90, 96.01-96.05, 96.7 |
| therapy/ventilator/intubation | |
| Erectile dysfunction | 607.84 |
| Major organ bleeding | 578.0, 578.1 |
| Muscle pain, myopathy, or | 728.8, 729.9, 791.3, 781.99 |
| rhabdomyolysis | |
| Injury and poisoning | 800-989 |
| Parkinson's disease | 332 |
| Huntington's disease | 333.4 |
| Mild cognitive impairment | 331.83 |
| Memory loss | 780.93 |
| Creutzfeldt-Jakob disease | 046.1 |
| Hypothyroidism | 243-244 |

Medication exposure may be defined by the British National Formulary (BNF) sections. Table 4 below includes an example drug list defined by the BNF. Each of the drugs in the drug lists may be further distinguished into subclasses based on drug names.

TABLE 4

| Drug class | BNF chapter |
|---|---|
| Corticosteroids | 1.5.2, 1.7.2, 3.2, 6.3, 8.2.2, 10.1.2, 11.4.1, 13.4 |
| H2 (histamine type 2)-receptor antagonists | 1.3.1 |
| Proton-pump inhibitors | 1.3.5 |
| Cardiac glycosides | 2.1.1 |
| Anti-arrhythmic drugs | 2.3.2 |
| Psychotropic drugs | 4.1, 4.2, 4.3, 4.4 |
| Antihypertensive drugs | 2.2, 2.4, 2.5.1, 2.5.2, 2.5.4, 2.5.5, 2.6.2 |
| Nitrates | 2.6.1 |
| Anticoagulants | 2.8.1, 2.8.2 |
| Antiplatelet drugs | 2.9 |
| Antidiabetic drugs | 6.1.1.1, 6.1.1.2, 6.1.2.1, 6.1.2.2, 6.1.2.3 |
| Lipid-modifying drugs | 2.12 |
| Nicotine replacement therapy | 4.10.2 |
| Oestrogen | 6.4.1 |
| Testosterone | 6.4.2 |
| Non-steroidal anti-inflammatory drugs | 10.1.1 |
| Thyroid hormones | 6.2.1 |
| Antithyroid drugs | 6.2.2 |

Table 5 below includes an example of Lipid-modifying drugs subclasses of the Lipid-modifying drug class identified in Table 4 above.

TABLE 5

| Subclass | Drug name |
|---|---|
| Statins | Atorvastatin, Fluvastatin, Lovastatin, Pravastatin, |
| | Rosuvastatin, Simvastatin |
| Fibrates | Bezafibrate, Clofibrate, Fenofibrate, Gemfibrozil |
| Niacin | Nicotinic acid, Nicotinate, Tredaptive, Acipimox |
| PCSK9 (Proprotein convertase | Alirocumab, Evolocumab |
| subtilisin/kexin type 9) inhibitors | |
| Cholesterol absorption inhibitors | Ezetimibe |
| Bile acid sequestrants | Cholestyramine |
| Omega-3 fatty acids | Maxepa |
| Vytorin | Vytorin |
| Others | Benfluorex, Probucol |

### Model Derivation

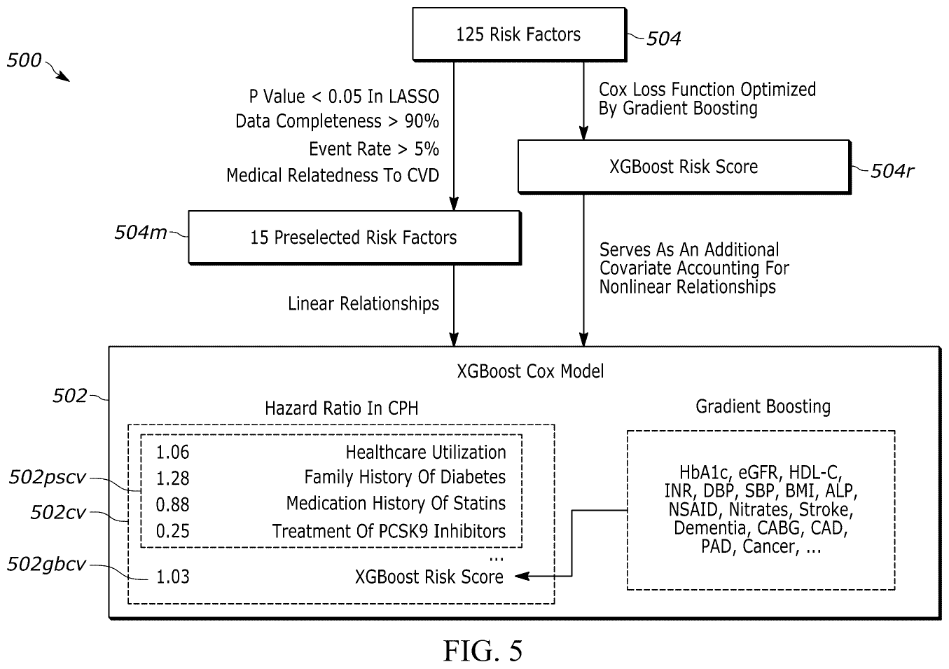

In some aspects, the ML model described herein comprises a hybrid statistical-ML model, which uses both statistical and machine learning algorithms to generate the ML model described herein. The design of the hybrid statistical-ML model is illustrated in FIG. 5. FIG. 5 illustrates an example ML model (e.g., ML model 502) and an example plurality of cardiovascular risk factors (e.g., cardiovascular risk factors 504) for training the ML model, in accordance with various embodiments disclosed herein. In the example of FIG. 5, ML model 502 comprises a machine learning model trained with, based on, or otherwise using a Cox-based model and the XGBoost gradient boosting algorithm. In the example of FIG. 5, ML model 502 is trained with data of a plurality of cardiovascular risk factors 504 specific to a population of given geographic region (e.g., China). While the example embodiment of FIG. 5 comprises 125 risk factors, it is to be understood that additional, fewer, or different numbers of risk factors may be used, and that the number of risk factors used is not limited to 125 risk factors.

In the example of FIG. 5, the plurality of cardiovascular risk factors (e.g., cardiovascular risk factors 504) is subdivided into a first training data subset and a second training data subset prior to training the ML model (e.g., ML model 502). The first training data subset comprises a preselected subset of cardiovascular risk factors (504m). In some aspects, the preselected risk factors are identified as highly predictive covariates with respect to predicting cardiovascular disease and/or have a higher level of data completeness, event rate occurrence, or medical values or other conditions related to CVD (e.g., including, by way of non-limiting example, values or conditions such as family history of disease, medication history, drug use, etc., and/or conditions or values associated with preselected risk variables indicated in Table 2 with “*” markings, and/or as otherwise described herein). For example, as shown in the example of FIG. 5, a preselected subset of cardiovascular risk factors (504m) is chosen corresponding to healthcare utilization, family history of diabetes, medication history of statins, and treatment of PCSK9 inhibitors (i.e., Proprotein convertase subtilisin/kexin type 9). In addition, in the embodiment of FIG. 5, such factors are identified as having P-values less than 0.5 (in LASSO), data completeness of 90% or greater, an event rate of 5 percent or more, and medical relatedness or correlation to CVD. Further, such factors have a linear relationship with a Cox proportional hazards (CPH) model, upon which the ML Model 502 is based otherwise implements. That is, in various aspects, the preselected subset of cardiovascular risk factors (504m) have a linear relationship with the ML model 502 where, for example, the ML model 502 defines one or more of the cardiovascular risk factors (504m) as having numeric weighted values relative to one another such that that an increase of a input to the model for a given cardiovascular risk factor impacts the output (e.g., a prediction) of the ML model 502 according to the respective weighted values across a linear predictive spectrum. As illustrated for Table 2, the preselected subset of cardiovascular risk factors (504m) may comprise one or more of values related to: age, sex, family history of diabetes, accident and emergency visits per year, aspartate transaminase, alanine aminotransferase, low-density lipoprotein cholesterol, neutrophil, statins, myocardial infarction, angina, revascularization, atrial fibrillation, hypertension, and/or user history of diabetes. In particular each of these factors are preselected covariates (e.g., preselected covariates 502pscv) in ML Model 502 having respective values of 1.06, 1.28, 0.88, and 0.25. More generally, a CPH model evaluates the effect of factors on survival. That is, each factor influences the rate of a particular event happening (e.g., death based on cardiovascular disease) at a particular point in time. This rate is commonly referred as the hazard rate. Predictor variables (or factors), such as covariates 502pscv, provide weighted values that influence the score or value as outputted by ML model 502, which is a CPH based model in the example of FIG. 5. In this way ML model 502, upon receiving as input user-specific cardiovascular data of a given user, can output a user-specific cardiovascular prediction of the user. In some aspects, the preselected subset of cardiovascular risk factors (504m) may be considered mandatory risk factors, e.g., where such risk factors are determined as highly predictive, and, therefore necessary for predicting a given disease (e.g., cardiovascular disease). However, it is to be understood that the preselected subset of cardiovascular risk factors (504m) may not always be mandatory, and, in some cases, one or more of the preselected subset of cardiovascular risk factors (504m) may be optional.

Further as shown for FIG. 5, a second training subset comprises a remaining subset of cardiovascular risk factors (504r). The remaining subset of cardiovascular risk factors (504r) may comprise all those risk factors not chosen as preselected risk factors, e.g., the preselected subset of cardiovascular risk factors (504m). The remaining subset of cardiovascular risk factors (504r) are also referred to herein as supplementary risk factors

As shown for FIG. 5, a gradient boosting algorithm (e.g., XGBoost) is applied to the remaining subset of the cardiovascular risk factors (504). In general, XGBoost (Extreme Gradient Boosting) is an optimized distributed gradient boosting software library or package designed for efficient and scalable training of machine learning models. XGBoost implements ensemble learning that combines the predictions of multiple weak models to produce an improved prediction. XGBoost, or gradient boosting in general, may be applied to large datasets and provide enhanced performance for machine learning tasks such as classification and regression. Application of XGBoost allows efficient handling of missing values such as those in remaining subset of cardiovascular risk factors (504r) without requiring significant pre-processing or data backfilling.

Applying the gradient boosting algorithm (e.g., XGBoost) to the data of the remaining subset of cardiovascular risk factors (504r) allows for generation of an additional covariate for use in the ML Model 502 and to account for a nonlinear relationship between such remaining subset of cardiovascular risk factors (504r) and the preselected subset of cardiovascular risk factors (504m). That is, in various aspects, remaining cardiovascular risk factors (504r) have a non-linear relationship with the ML model 502 where, for example, the ML model 502 defines one or more of such risk factors (504r) as an overall value or score that can be used as an input to the model to impact the output (e.g., a prediction) of the ML model 502 according to such overall value or score. As shown in the example of FIG. 5, gradient boosting (e.g., XGBoost) is applied to various remaining cardiovascular risk factors (504r) including HbA1c, eGFR, HDL-C, INR, DBP, SBP, BMI, ALP, NSAID, nitrates, stroke, dementia, CABG, CAD, PAD, cancer, and/or others as described herein. The application or otherwise execution of the gradient boosting (e.g., XGBoost) algorithm to the remaining cardiovascular risk factors (504r) creates a gradient boost covariate 502gbcv, which may define a risk score (e.g., an XGBoost risk score) that defines or accounts for the remaining or otherwise supplementary data expressed by the data of the remaining cardiovascular risk factors (504r).

More generally, with respect to FIG. 5, a feature selection procedure was first applied to all available risk variables (e.g., 125 cardiovascular risk factors) to identify preselected risk variables (504m) for model interpretability. In some embodiments, and especially with respect to the identification of preselected risk variables, and for improved statistical reliability and clinical utility, risk variables with missing rates below 10% (e.g., clinical laboratory tests) and an event rate above 5% (e.g., disease and medication history) were passed (i.e., allowed) for feature selection. In such aspects, at least a portion of the preselected subset of cardiovascular risk factors (504m) comprises imputed data generated to replace missing values.

In some aspects, with respect to the preselected subset of cardiovascular risk factors (504m), multivariate imputation with chained equations (MICE) can be used to generate an imputed dataset to replace any missing values, e.g., of clinical laboratory tests. Generally, MICE can be implemented to address issues with missing or incomplete data, which can occur in large datasets comprising, for example, hundreds of variables of varying types. MICE is an algorithm where a series of regression models are run whereby each variable with missing data is modeled conditional upon the other variables in the data. In particular, each variable can be modeled according to its distribution, with, for example, binary variables modeled using logistic regression and continuous variables modeled using linear regression. MICE can be implemented, for example, to high-dimensional datasets with various missing patterns to replace any missing values and/or to otherwise complete a training dataset.

As used with respect to the embodiments herein, for example for FIG. 5, covariates 502pscv may comprise at least some missing values. In an example implementation of MICE, each variable of covariates 502pscv is first imputed using, e.g. mean imputation, temporarily setting any missing value equal to the mean observed value for that variable. The imputed mean values of one of the covariates (e.g., healthcare utilization) are then set back to a missing state or destination. In a further step, a linear regression of healthcare utilization is predicted by the other covariates 502pscv by executing or running cases where healthcare utilization was observed. In a further step, predictions of the missing values of healthcare utilization are obtained from that regression equation and imputed. At this point, healthcare utilization does not have any missing values (no missingness). These steps could then be repeated for the other covariates 502pscv having missing values. This entire process of iterating through each of the covariates 502pscv with missing or otherwise incomplete values is repeated until convergence or completeness of the dataset is reached. That is, in the imputation example, the observed data and the final set of imputed values are then constitute a complete data set for the preselected subset of cardiovascular risk factors (504m), which can be used to train the ML model 502, for example, as described herein.

Further, in some aspects, the remaining subset of cardiovascular risk factors (504r) are not imputed. Non-imputed data may comprise raw data. Use of raw data for remaining subset of cardiovascular risk factors (504r) allows the invention herein to operate with reduced memory data storage requirements, while still allowing the ML model to be highly predictive.

With further reference to FIG. 5, and as described above, ML model 502 can comprise, implement, or be based on a Cox proportional hazards model (CPH). In some aspects, the CPH model may utilize a least absolute shrinkage and selection operator (LASSO) regularization to filter the preselected subset of cardiovascular risk factors (504m) to those statistically significant (p value<005) risk variables. More generally, a Cox proportional hazards model can be implemented as a multivariate statistical model for survival analysis where its regression coefficients can be interpreted as hazard ratios (e.g., which can be easily understood by clinicians for better decision-making). Further, the LASSO can be implemented as a feature selection method for selecting a representative and independent set of risk variables for providing reliable downstream manual prioritization. Such preselected subset of cardiovascular risk factors (504m) (e.g., risk variables) may also be determined or otherwise filtered based on clinical relevance to ensure the final set of risk variables are comprehensive and relevant to CVD prognosis. As shown for FIG. 5, preselected subset of cardiovascular risk factors (504m) (e.g., risk variables) are included in the ML model 502 as linear covariates.

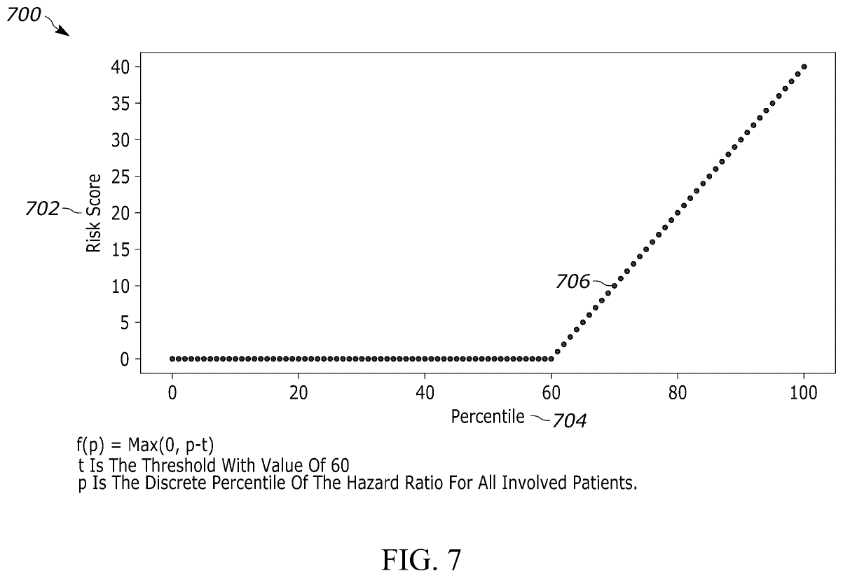

A gradient boosting algorithm (e.g., the XGBoost algorithm) may be implemented to yield improved model performance. The gradient boosting algorithm allows measurement and integration of complex effects from all risk variables in EHR related data. The gradient boosting algorithm can be implemented to address real-world EHR data issues where cohorts of data can be highly heterogeneous in form, distribution, and especially completeness. For example, a gradient boosting algorithm (e.g., the XGBoost algorithm) can be implemented with P-CARDIAC to fit a tree-ensembled hazard ratio based on all risk variables (e.g., as described herein). Such implementation solves data training issues inherent with most ML methods, which typically require complete data sets the lack thereof can cause huge imputation bias in high-dimensional data sets. Further, implementation of a gradient boosting algorithm (e.g., the XGBoost algorithm) can provide a gradient boosting decision tree method, which can be applied to heterogeneous tabular data. Moreover, a gradient boosting algorithm (e.g., the XGBoost algorithm) can be implemented even though missing values may exist within a given dataset. For example, for one implementation, to cancel out non-linear distribution bias in the raw output of a gradient boosting algorithm (e.g., the XGBoost algorithm), the raw output hazard ratio can be first mapped to discrete percentiles, which can improve model calibration performance. To balance the significance between the XGBoost risk score and other risk variables in a given model, the percentiles can be mapped onto a hinge loss-like function (e.g., as shown for FIG. 7). In such implementation, the AI model (e.g., P-CARDIAC full model) with all 125 risk variables can be a CPH model with ridge regularization regressed on the risk variables and the XGBoost risk score. In various aspects, ridge regularization can be implemented as a stabilizer of regression coefficients, which can provide reliable estimates of the hazard ratios of the risk variables.

Applying gradient boosting enhances model performance. That is the full ML model, using both preselected subset of cardiovascular risk factors (504m) and remaining cardiovascular risk factors (504r) yields a more accurate predictive model. Thus, as shown for FIG. 5, the measurement and integration of complex effects from all risk variables in the electronic health records (EHR) is important for enhanced model accuracy. Real world EHR data, as found in the data for the cohorts described herein, are highly heterogeneous in form, distribution, and especially completeness. Therefore, application of a gradient boosting algorithm against such heterogeneous data (e.g., data of the remaining cardiovascular risk factors (504r) yields enhanced model predictive accuracy. That is, in some aspects, a gradient boosting algorithm is implemented or applied a gradient boosting algorithm to the second training subset of the remaining subset of cardiovascular risk factors (504r) to enhance the Cox proportional hazards model. For example, XGBoost, a gradient boosting decision tree method, can be applied to the Cox model developed with the preselected subset of cardiovascular risk factors (504m) to generate an ML model (e.g., a full P-CARDIAC model) to fit a tree-ensembled hazard ratio based on all risk variables for better dealing with heterogeneous tabular data. An example implementation gradient boosting a Cox proportional hazards model (e.g., model 502) using XGBoost is described as follows. Generally, the Cox model is expressed by the hazard function denoted by h(t). The hazard function defined by the Cox model can be interpreted as the risk of dying (or other event of interest happening) at time t:

$$ln ( h ( t ) ) = ln ( h 0 ( t ) ) + < w , x > \qquad ( 1 )$$

In the above equation 1, x is a vector in the mathematical domain of real numbers (Rd) representing the features; w is a vector consisting of d coefficients, each corresponding to a feature; the notation [symbol] , [symbol] is the usual dot product in Rd; ln(·) is the natural logarithm; and the term h0(t) is the baseline hazard.

In the example of FIG. 5, implementation of XGBoost enhances the Cox proportional hazards (CPH) model as defined by the following equation to make Cox operate with gradient boosting:

$$ln ( h ( t ) ) = ln ( h 0 ( t ) ) + T ( x ) \qquad ( 2 )$$

In equation 2 above, T(x) represents the output from a decision tree ensemble, given input x. Use of XGBoost maximizes the (log) likelihood by fitting an accurate tree ensemble T(x). Thus, in some aspects, ML model 502 can implement an enhanced CPH model as defined by equation 2.

Additional modifications to the ML model 502, or its output, can also be performed to enhance the predictive output (e.g., a user-specific cardiovascular prediction comprising a cardiovascular risk score of given user) of ML model 502. For example, to cancel out non-linear distribution bias in the raw output of XGBoost, a raw output hazard ratio can be mapped to discrete percentiles. Such elimination of non-linear distribution bias can increase model calibration performance of ML model 502.

Further, to balance the significance between the gradient boost covariate 502gbcv (e.g., XGBoost risk score as shown for FIG. 5) and other risk variables in the final model (e.g., preselected covariates 502pscv), the percentiles can also be mapped onto a hinge loss-like function, as shown for example, for FIG. 7. FIG. 7 illustrates an example function of a threshold value defining a risk score as output by the ML model as described for FIG. 5. In the example of FIG. 7, the function illustrates the design of hinge loss-like function, which may be defined as follows:

$$f ( p ) = max ( 0 , p ‐ t ) \qquad ( 3 )$$

In equation 3 above, t is the threshold (e.g., with a value of 60 in the example of FIG. 7), and p is the discrete percentile of the hazard ratio for all involved patients. With reference to FIG. 7, and as shown by slope 706, ML model 502 is configured to output a risk score 702 of a value of 0 across percentile 704 values from 0-60 and output a value that is linear across values 60-100. Such values may be output by ML model 502, where the hinge loss-like function reconfigures ML model 502 to output a threshold-based risk depending on where value t (the threshold) is set or otherwise configured for ML model 502. In this way, ridge regularization can be used as a stabilizer of regression coefficients, which provides reliable estimates of the hazard ratios of the risk variables.

### Model Validation

In the examples of this disclosure, internal consistency of model performance was evaluated on the derivation cohort by 100 repeats of 10-fold cross-validation. Model performance of the ML model 502 (e.g., the P-CARDIAC model), TRS-2° P, and SMART2 was compared using the validation cohorts with 1,000 bootstrap replicates.

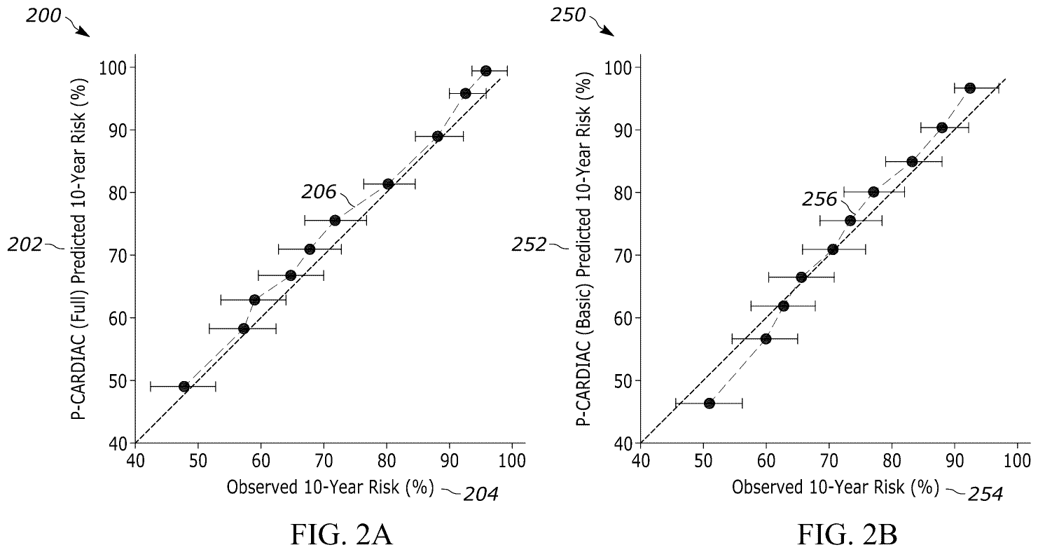

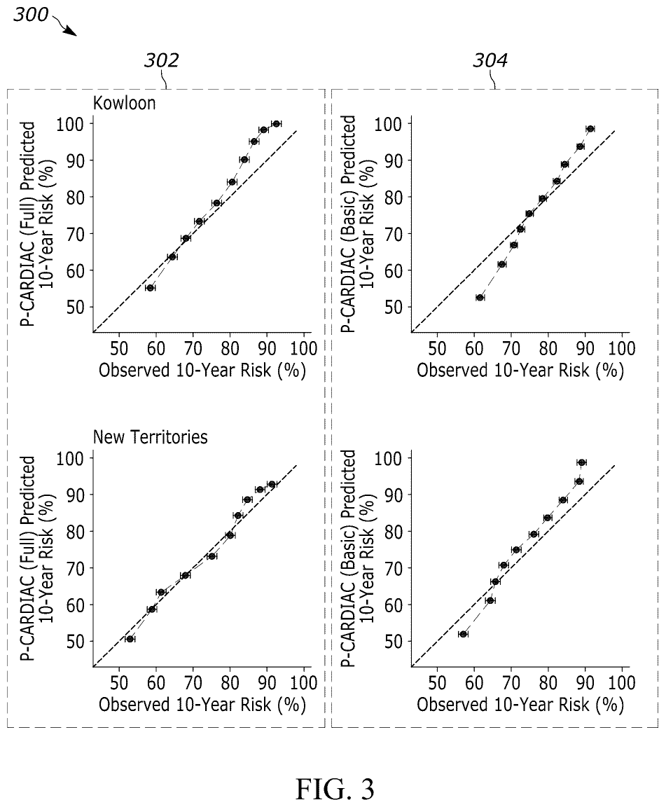

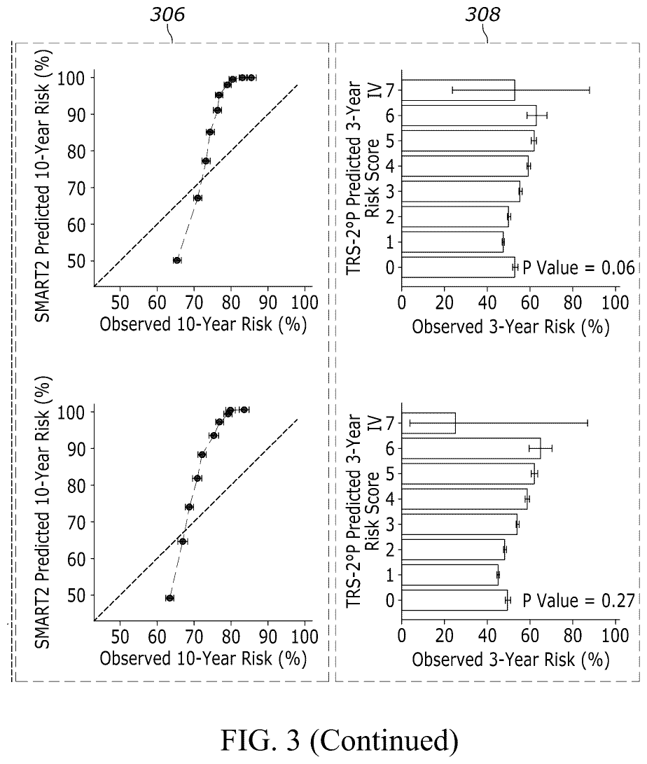

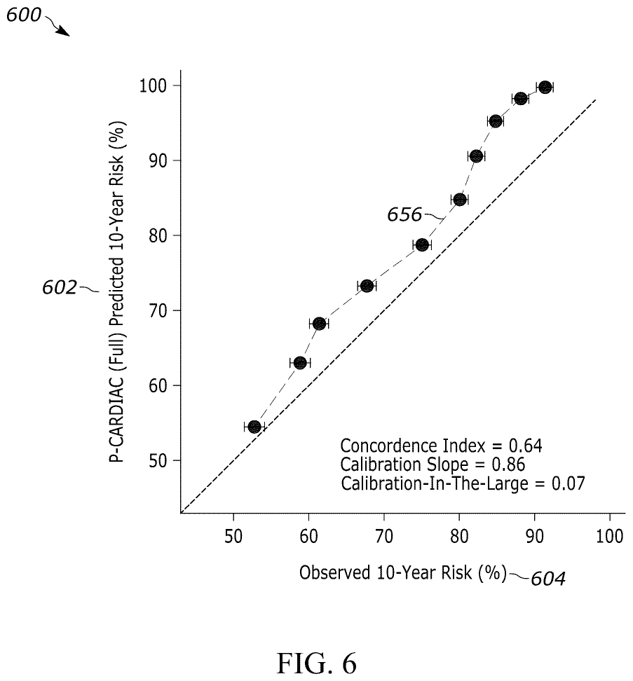

In some aspects, calibration performance can be assessed graphically by categorizing patients into deciles of predicted 10-year CVD risk and plotting mean 10-year predicted risk against observed 10-year risk. In the present example, the observed 10-year risk was obtained by the Kaplan-Meier method. Means and confidence intervals of Harrell's C-statistic, calibration-in-the-large, and calibration slope were calculated. The calibration slope was the slope of linear regression of the observed risk against the predicted risk of each decile. Recalibration was performed if there was overall overestimation or underestimation observed in the calibration curves. For example, recalibration is demonstrated with respect to FIGS. 2A, 3, and FIG. 6. That is, FIG. 6 illustrates a calibration plot 600 for a full ML model (e.g., full P-CARDIAC model) before recalibration as shown for FIGS. 2A and/or 3 was performed, in accordance with various embodiments disclosed herein. With reference to FIG. 6, the full ML model (e.g., full P-CARDIAC model) may comprise a machine learning model trained on a plurality of cardiovascular risk factors (e.g., cardiovascular risk factors 504) subdivided into a first training data subset and a second training data subset, where the first training data subset comprises a preselected subset of cardiovascular risk factors (e.g., cardiovascular risk factors 504m), and where the second training subset comprises a remaining subset of cardiovascular risk factors (e.g., cardiovascular risk factors 504r). As shown in the example of FIG. 6, the full ML model (even before recalibration) outputs a predicted 10-year risk percentage (602), which is compared against an observed 10-year risk percentage (604). As illustrated for FIG. 6, the predicted-to-observed plot 656 of the full ML model plot 600 (before recalibration) demonstrates accuracy with respect to the calibration slope, where the concordance index has a value of 0.64, the calibration slope has a value of 0.86, and the calibration-in-the large has a value of 0.07. The full ML model (e.g., full P-CARDIAC model) of FIG. 6 is an example of the full ML model on the New Territories cohort before recalibration. The full ML model may be recalibrated to improve its accuracy as shown and described for example in FIGS. 2A and 3 herein.

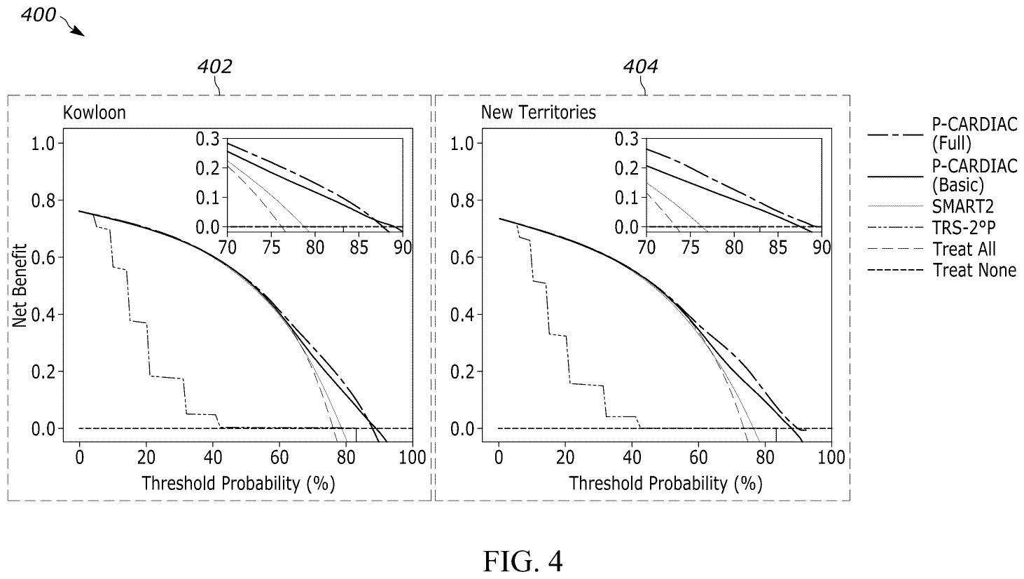

In addition, with respect to model curve review, decision curve analysis was used to estimate the effect of different treatment options across different threshold risks. Such implementation can identify the range of threshold risks where the model has clinical value (with positive net benefit) and the magnitude of the clinical value. For example, in some aspects, the ML model is further trained with data defining one or more threshold risks, where each threshold risk defines a magnitude of a clinical health benefit to a user of the geographic region. This is shown and described, for example, with respect to FIG. 4 herein. As a further example, in one aspect, first a threshold probability (pt) can be chosen to define when a patient is positive. Second, x=1 if the patient had a predicted probability from the model≥pt (the threshold probability) and x=0 otherwise; s(t) can be the Kaplan-Meier survival probability at a chosen landmark time t, and N can be the number of subjects in a given data set, where the number of true positives (TP)=[1−(s(t)|x=1)]×P(x=1)×N and the false positives (FP)=(s(t)|x=1)×P(x=1)×N. A net benefit=TP/N−FP/N×[pt/(1−pt)] can be calculated, and the above calculation can be repeated for a reasonable range of threshold probabilities. All steps for each model can be repeated as well as default strategies for treat-all and/or treat-none as if the result is positive. Generally, the model with higher net benefits across a larger range of threshold risks is the preferred model. Decision curve analysis can be used to describe and compare the 10-year clinical value of P-CARDIAC, TRS-2° P, and SMART2 on the two validation cohorts. TRS-2° P has proposed the specific 3-year risk regarding different risk scores, and the predicted 3-year risk is extrapolated to a 10-year risk by multiplying the ratio of the corresponding Kaplan-Meier estimated risks for each of the two cohorts.

### Results

### Study Cohorts

An exemplary flowchart of patient selection and related cohorts is illustrated in FIG. 1.

For the derivation cohort, 221,258 patients aged 18 or above were identified with lipid test records between 1 Jan. 2004 and 31 Dec. 2019. 172,459 patients were excluded from the cohort who had no diagnosis record of CVD or died of the first CVD event on the same date. Overall, 48,799 patients were included in the derivation cohort.

For the validation cohorts, a cohort of 2 million patients aged 35 or above was identified with blood pressure records in the HA between 2005 and 2019. 1,679,150 patients who had no diagnosis record of CVD or died of the first CVD event on the same date was excluded. 60,645 patients were excluded without healthcare utilization records or with the most frequently visited healthcare facility at Hong Kong Island. Overall, 119,672 patients were included in the New Territories cohort, and 140,533 patients were included in the Kowloon cohort.

### Incidence Rates of CVD and Baseline Characteristics

Table 6 below shows patient characteristics with event rates of CVD across three cohorts. The event rate per 1000 person-years was 219 to 241, while the median estimated 10-year event rate was 71-7-76-1%, respectively. During a median follow-up of 0-3 to 1-0 year, 55-64% of patients had cardiovascular disease recurrences. Regarding the composition of incident CVD events, coronary heart disease (CHD) was identified as the most common, with composition around 61-65%, of which MI had a ratio of approximately 9-10%. Stroke was the second most common outcome with a ratio of approximately 33-39%. The ratio of peripheral arterial disease (PAD) was around 3-4%.

TABLE 6

| | Hong Kong Island (Hong | | | | | |
|---|---|---|---|---|---|---|
| | Kong West Cluster) | | Kowloon | | New Territories | |
| Participants | 48,799 | | 140,533 | | 119,672 | |
| Incident cardiovascular events | 31,100 | (64%) | 80,498 | (57%) | 65,687 | (55%) |
| Coronary heart disease | 20,167 | (65%) | 49,754 | (62%) | 39,807 | (61%) |
| Myocardial infarction | 3,231 | (10%) | 7,341 | (9%) | 5,773 | (9%) |
| Stroke | 10,394 | (33%) | 30,342 | (38%) | 25,413 | (39%) |
| Peripheral artery disease | 1,102 | (4%) | 2,188 | (3%) | 1,826 | (3%) |
| Revascularization | 4,135 | (13%) | 5,396 | (7%) | 4,447 | (7%) |
| *Fatal events | 964 | (3%) | 4,544 | (6%) | 3,246 | (5%) |
| Total person-years observed | 141,829 | | 334,053 | | 293,269 | |
| Event rate per 1000 person- | 219 | | 241 | | 224 | |
| years | | | | | | |
| **Follow-up (years) | 0.3 | (0.0-13.5) | 0.9 | (0.0-10.4) | 1.0 | (0.0-10.5) |
| ***10-year event rate (%) | 71.7 | (71.3-72.2) | 76.1 | (75.8-76.5) | 73.3 | (72.9-73.7) |

All data in n (%) or median (interquartile range) unless indicated otherwise. All subtypes of incidence events in the Kowloon and New Territories cohorts were significantly different (p value < 0.05) compared to the Hong Kong Island (Hong Kong West Cluster) under Chi-square test. Event rate was the incident event divided by total person-years of each cohort.
*Deaths within 28 days after recurrent cardiovascular event.
**Median (5th/95th percentile).
***Mean (95% confidence interval), estimated by Kaplan-Meier method.

All subtypes of incidence events in the derivation cohort had significantly different distribution from the validation cohorts. The proportion of total CVD events was higher. The proportion of CHD, MI, PAD, and revascularization was higher, while the proportion of stroke and fatal events were lower. Table 7 shows the baseline characteristics of the risk variables across three cohorts, e.g., for the preselected factors (504m).

TABLE 7

| Hong Kong Island | | |
|---|---|---|
| (Hong Kong West | | |
| Cluster) | Kowloon | New Territories |

General [n (%), or median (interquartile range)]

| | | | | | | |
|---|---|---|---|---|---|---|
| Age (years) | 69 | (59-78) | 73 | (63-82) | 71 | (61-80) |
| Female | 18,948 | (39%) | 61,101 | (43%) | 50,187 | (42%) |
| Male | 29,851 | (61%) | 79,432 | (57%) | 69,485 | (58%) |
| Accident and emergency visits | 0.6 | (0.0-0.7) | 0.9 | (0.5-1.1) | 0.9 | (0.6-1.2) |
| per year | | | | | | |

Clinical laboratory tests [median (interquartile range, proportion of missing data)]

| | | | | | | |
|---|---|---|---|---|---|---|
| Low-density lipoprotein | 2.5 | (1.9-3.1, 0%) | 2.6 | (2.0-3.3, 5%) | 2.6 | (2.0-3.3, 4%) |
| cholesterol (mmol/L) | | | | | | |
| Neutrophil (10\hat{ }9/L) | 4.9 | (3.7-6.8, 2%) | 5.3 | (3.9-7.8, 3%) | 5.3 | (3.9-7.7, 2%) |
| Aspartate transaminase: | 1.1 | (0.8-1.6, 1%) | 1.3 | (0.9-1.9, 37%) | 1.3 | (0.8-2.2, 80%) |
| alanine aminotransferase ratio | | | | | | |

Disease and medication history [n (%)]

| | | | | | | |
|---|---|---|---|---|---|---|
| Statins | 12,801 | (26%) | 47,278 | (34%) | 42,127 | (35%) |
| Hypertension | 30,583 | (63%) | 109,374 | (78%) | 92,568 | (77%) |
| Diabetes | 12,388 | (25%) | 43,096 | (31%) | 37,217 | (31%) |
| Atrial fibrillation | 4,248 | (9%) | 13,920 | (10%) | 11,251 | (9%) |
| Myocardial infarction | 5,361 | (11%) | 23,626 | (17%) | 18,162 | (15%) |
| Angina | 3,548 | (7%) | 10,389 | (7%) | 7,126 | (6%) |
| Revascularization | 6,839 | (14%) | 6,199 | (4%) | 6,455 | (5%) |
| Family history of diabetes | 4,878 | (10%) | 17,278 | (12%) | 15,613 | (13%) |

Drug use [n (%)]

| | | | | | | |
|---|---|---|---|---|---|---|
| Antihypertensive drugs | 38,851 | (80%) | 121,287 | (86%) | 101,353 | (85%) |
| Antidiabetic drugs | 12,995 | (27%) | 44,081 | (31%) | 37,644 | (31%) |
| Antiplatelet drugs | 35,575 | (73%) | 116,263 | (83%) | 99,051 | (83%) |
| Statins | 31,452 | (64%) | 90,856 | (65%) | 84,260 | (70%) |
| Fibrates | 1,201 | (2%) | 3,491 | (2%) | 2,402 | (2%) |
| Niacin | 65 | (0%) | 16 | (0%) | 20 | (0%) |
| PCSK9 (Proprotein convertase | 30 | (0%) | 22 | (0%) | 48 | (0%) |
| subtilisin/kexin type 9) | | | | | | |
| inhibitors | | | | | | |
| Cholesterol absorption | 666 | (1%) | 853 | (1%) | 1,102 | (1%) |
| inhibitors | | | | | | |

All risk variables in the Kowloon and New Territories cohorts were significantly different (p value < 0.05) compared to the Hong Kong Island (Hong Kong West Cluster) under Chi-square test (categorical risk variables) or in T-test (numerical risk variables).

Table 8 shows the baseline characteristics of the risk variables across three cohorts, e.g., for the remaining (e.g., supplementary) set of variables (504r).

TABLE 8

| | | |
|---|---|---|
| Hong Kong Island | | |
| (Hong Kong West | | |
| Cluster) | Kowloon | New Territories |

Clinical laboratory tests [median (interquartile range, proportion of missing data)]

| | | | | | | |
|---|---|---|---|---|---|---|
| Aspartate transaminase | 25.0 | (20.0-33.0, 1%) | 24.0 | (18.0-35.0, 37%) | 27.0 | (20.0-45.0, 80%) |
| (IU/L) | | | | | | |
| Alanine aminotransferase | 23.0 | (16.0-34.0, 1%) | 19.0 | (14.0-28.9, 0%) | 20.0 | (14.0-30.0, 0%) |
| (IU/L) | | | | | | |
| Haemoglobin A1c (%) | 6.1 | (5.7-6.9, 24%) | 6.1 | (5.7-6.8, 16%) | 6.1 | (5.7-6.8, 14%) |
| Creatine kinase (IU/L) | 109.0 | (70.0-196.0, 14%) | 115.0 | (71.0-212.0, 11%) | 113.0 | (72.0-201.1, 10%) |
| Prothrombin time (second) | 11.7 | (11.0-12.5, 7%) | 11.6 | (10.8-12.5, 7%) | 11.4 | (10.7-12.2, 8%) |
| Potassium (mmol/L) | 4.0 | (3.7-4.3, 0%) | 4.0 | (3.7-4.4, 0%) | 4.0 | (3.6-4.3, 0%) |
| Estimated glomerular | 69.6 | (52.4-84.0, 33%) | 70.0 | (53.5-85.0, 24%) | 73.0 | (57.0-87.0, 29%) |
| filtration rate (mL/min/1.73 | | | | | | |
| m\hat{ }2) | | | | | | |
| Triglycerides (mmol/L) | 1.2 | (0.9-1.6, 0%) | 1.2 | (0.9-1.7, 4%) | 1.2 | (0.9-1.7, 4%) |
| Basophil (10\hat{ }9/L) | 0.0 | (0.0-0.0, 2%) | 0.0 | (0.0-0.0, 3%) | 0.0 | (0.0-0.1, 2%) |
| Arterial partial pressure of | 11.5 | (6.8-16.1, 51%) | 8.8 | (4.6-14.3, 37%) | 9.0 | (4.7-14.0, 43%) |
| oxygen (kPa) | | | | | | |
| Albumin (g/L) | 41.0 | (37.0-44.0, 1%) | 39.0 | (35.0-42.0, 0%) | 39.4 | (36.0-42.3, 0%) |
| International normalized | 1.0 | (1.0-1.1, 7%) | 1.0 | (1.0-1.1, 7%) | 1.0 | (1.0-1.1, 8%)* |
| ratio | | | | | | |
| Diastolic blood pressure | 73.0 | (65.0-82.0, 46%) | 74.0 | (66.0-84.0, 0%) | 75.0 | (67.0-85.0, 0%) |
| (mmHg) | | | | | | |
| Bicarbonate (mmol/L) | 23.9 | (21.2-26.4, 5%) | 24.0 | (21.0-26.6, 31%) | 23.9 | (21.0-26.5, 39%) |
| Glucose (mmol/L) | 5.7 | (5.1-6.8, 4%) | 5.7 | (5.1-6.9, 4%) | 5.7 | (5.1-6.9, 3%) |
| Erythrocyte sedimentation | 45.0 | (20.0-85.0, 54%) | 37.0 | (19.0-69.0, 50%) | 34.0 | (16.0-65.0, 48%) |
| rate (mm/hr) | | | | | | |
| Free thyroxine (pmol/L) | 16.0 | (13.9-18.1, 51%) | 14.3 | (12.5-16.5, 56%) | 14.8 | (12.8-17.2, 56%) |
| Troponin I (ng/ml) | 0.0 | (0.0-0.1, 53%) | 0.0 | (0.0-0.1, 41%) | 0.0 | (0.0-0.1, 54%) |
| Bilirubin (umol/L) | 9.2 | (7.0-13.0, 1%) | 10.0 | (7.0-14.2, 0%) | 10.0 | (7.0-14.0, 0%) |
| C-reactive protein (mg/dL) | 1.3 | (0.3-5.8, 53%) | 2.0 | (0.4-7.5, 38%) | 1.2 | (0.3-5.7, 36%) |
| Total cholesterol (mmol/L) | 4.3 | (3.6-5.1, 0%) | 4.5 | (3.8-5.3, 4%) | 4.5 | (3.8-5.3, 4%) |
| Blood pH | 7.4 | (7.4-7.5, 47%) | 7.4 | (7.4-7.4, 35%) | 7.4 | (7.4-7.4, 43%) |
| Systolic blood pressure | 135.0 | (122.0-149.0, 46%) | 139.0 | (125.0-155.0, 0%) | 138.0 | (124.0-154.0, 0%) |
| (mmHg) | | | | | | |
| Thyroid stimulating | 1.3 | (0.9-2.1, 30%) | 1.3 | (0.8-2.1, 15%) | 1.4 | (0.9-2.1, 15%) |
| hormone (mIU/L) | | | | | | |
| Lymphocyte (10\hat{ }9/L) | 1.6 | (1.2-2.1, 2%) | 1.5 | (1.1-2.1, 3%) | 1.6 | (1.1-2.1, 2%)* |
| Creatinine (umol/L) | 88.0 | (73.0-109.0, 0%) | 86.0 | (70.0-109.0, 0%)* | 84.0 | (69.0-104.0, 0%) |
| Platelet (10\hat{ }9/L) | 223.0 | (184.0-268.0, 2%) | 222.0 | (181.0-269.0, 1%)* | 222.0 | (182.0-268.0, 1%)* |
| Red blood cell (10\hat{ }12/L) | 4.4 | (4.0-4.8, 2%) | 4.4 | (3.9-4.8, 1%) | 4.4 | (4.0-4.8, 1%) |
| High-density lipoprotein | 1.2 | (0.9-1.4, 0%) | 1.2 | (1.0-1.5, 5%) | 1.2 | (1.0-1.5, 4%) |
| cholesterol (mmol/L) | | | | | | |
| Body mass index (kg/m\hat{ }2) | 24.7 | (22.2-27.3, 62%) | NA | (NA, 100%) | NA | (NA, 100%) |
| Calcium (mmol/L) | 2.3 | (2.2-2.4, 13%) | 2.3 | (2.2-2.4, 5%) | 2.3 | (2.2-2.4, 4%) |
| White blood cell (10\hat{ }9/L) | 7.4 | (6.0-9.4, 2%) | 8.0 | (6.4-10.4, 1%) | 7.9 | (6.3-10.2, 1%) |
| Alkaline phosphatase (IU/L) | 73.6 | (61.0-90.0, 1%) | 75.0 | (62.0-92.0, 0%) | 74.0 | (61.0-91.0, 0%) |
| Sodium (mmol/L) | 141.0 | (138.0-143.0, 0%) | 139.8 | (137.0-141.9, 0%) | 139.9 | (137.3-141.6, 0%) |
| Eosinophil (10\hat{ }9/L) | 0.1 | (0.1-0.2,2%) | 0.1 | (0.0-0.2, 3%) | 0.1 | (0.0-0.2, 2%) |
| Haemoglobin (g/dL) | 13.4 | (12.1-14.5, 2%) | 13.1 | (11.7-14.3, 1%) | 13.3 | (11.9-14.4, 1%) |
| Monocyte (10\hat{ }9/L) | 0.4 | (0.3-0.6, 2%) | 0.5 | (0.4-0.7, 3%) | 0.5 | (0.4-0.7, 2%) |

Disease history [n (%)]

| | | | | | | |
|---|---|---|---|---|---|---|
| Congestive heart failure | 3,726 | (8%) | 13,824 | (10%) | 10,715 | (9%) |
| Stroke | 16,985 | (35%) | 62,743 | (45%) | 54,163 | (45%) |
| Thyroid disease | 1,019 | (2%) | 3,455 | (2%) | 2,720 | (2%) |
| Arrhythmia and conduction | 5,956 | (12%) | 21,115 | (15%) | 16,378 | (14%) |
| disorders | | | | | | |
| Obesity | 633 | (1%) | 3,460 | (2%) | 3,202 | (3%) |
| Coronary heart disease | 30,662 | (63%) | 76,562 | (54%) | 64,128 | (54%) |
| Hypothyroidism | 433 | (1%) | 1,695 | (1%) | 1,421 | (1%) |
| Cardiac wall/valve/shunt | 205 | (0%) | 322 | (0%) | 224 | (0%) |
| replacement/repairment | | | 5,589 | (5%) | | |
| Oxygen | 1,359 | (3%) | 8,518 | (6%) | | |
| therapy/ventilator/intubation | | | | | | |
| Asthma | 740 | (2%) | 2,413 | (2%) | 1,765 | (1%) |
| Injury and poisoning | 5,164 | (11%) | 20,980 | (15%) | 17,579 | (15%) |
| Alcohol user | 313 | (1%) | 1,008 | (1%) | 914 | (1%) |
| Dyslipidaemia | 7,047 | (14%) | 34,258 | (24%) | 28,764 | (24%) |
| Cardiomyopathy | 407 | (1%) | 661 | (0%) | 653 | (1%) |
| Parkinson's disease | 303 | (1%) | 1,134 | (1%) | 880 | (1%) |
| Defibrillator insertion | 154 | (0%) | 146 | (0%) | 68 | (0%) |
| Major organ bleeding | 187 | (0%) | 727 | (1%) | 604 | (1%) |
| Severe mental illness | 3,929 | (8%) | 16,181 | (12%) | 13,874 | (12%) |
| Dementia | 1,810 | (4%) | 9,041 | (6%) | 7,083 | (6%) |
| Pacemaker implantation | 635 | (1%) | 1,296 | (1%) | 1,241 | (1%) |
| Liver disease | 1,187 | (2%) | 5,385 | (4%) | 3,874 | (3%) |
| Chronic obstructive | 1,527 | (3%) | 7,240 | (5%) | 5,793 | (5%) |
| pulmonary disease | | | | | | |
| Cancer | 3,328 | (7%) | 9,991 | (7%) | 7,517 | (6%) |
| Rheumatoid arthritis | 334 | (1%) | 867 | (1%) | 659 | (1%) |
| Renal disease | 3,268 | (7%) | 12,455 | (9%) | 9,425 | (8%) |
| Smoker | 274 | (1%) | 2,776 | (2%) | 895 | (1%) |
| Chronic kidney disease | 1,798 | (4%) | 6,259 | (4%) | 4,450 | (4%) |
| Muscle pain or myopathy or | 137 | (0%) | 673 | (0%) | 449 | (0%) |
| rhabdomyolysis | | | | | | |
| Dialysis | 1,357 | (3%) | 5,215 | (4%) | 3,479 | (3%) |
| Creutzfeldt-Jakob disease | 3 | (0%) | 2 | (0%) | 1 | (0%) |
| Cardioversion | 31 | (0%) | 3 | (0%) | 4 | (0%) |
| Nephrotic syndrome | 189 | (0%) | 767 | (1%) | 562 | (0%) |
| Coronary artery bypass | 7 | (0%) | 17 | (0%) | 2 | (0%) |
| graft | | | | | | |
| Systemic lupus | 117 | (0%) | 169 | (0%) | 133 | (0%) |
| erythematosus | | | | | | |
| Heart transplantation | 5 | (0%) | 10 | (0%) | 5 | (0%) |
| Peripheral artery disease | 1,475 | (3%) | 3,244 | (2%) | 2,770 | (2%) |
| Migraine | 51 | (0%) | 143 | (0%) | 147 | (0%) |
| Down's syndrome | 4 | (0%) | 16 | (0%) | 7 | (0%) |
| Family history of | 239 | (0%) | 1,636 | (1%) | 1,778 | (1%) |
| cardiovascular disease | | | | | | |

Medication history [n (%)]

| | | | | | | |
|---|---|---|---|---|---|---|
| Antihypertensive drugs | 25,986 | (53%) | 102,429 | (73%) | 87,215 | (73%) |
| Antidiabetic drugs | 8,923 | (18%) | 36,480 | (26%) | 31,860 | (27%) |
| Antiplatelet drugs | 16,882 | (35%) | 61,991 | (44%) | 51,168 | (43%) |
| Non-steroidal anti- | 14,018 | (29%) | 58,562 | (42%) | 57,593 | (48%) |
| inflammatory drugs | | | | | | |
| Corticosteroids | 15,391 | (32%) | 68,034 | (48%) | 58,831 | (49%) |
| Proton-pump inhibitors | 8,666 | (18%) | 33,265 | (24%) | 26,564 | (22%) |
| H2-receptor antagonists | 16,454 | (34%) | 76,727 | (55%) | 68,887 | (58%) |
| Anticoagulants | 2,994 | (6%) | 7,329 | (5%) | 7,001 | (6%) |
| Nicotine replacement | 386 | (1%) | 966 | (1%) | 1,920 | (2%) |
| therapy | | | | | | |
| Antiarrhythmic drugs | 1,269 | (3%) | 3,194 | (2%) | 2,462 | (2%) |
| Antithyroid drugs | 325 | (1%) | 1,469 | (1%) | 1,290 | (1%) |
| Oestrogen | 358 | (1%) | 652 | (0%) | 534 | (0%) |
| Psychotropic drugs | 7,013 | (14%) | 24,838 | (18%) | 23,931 | (20%) |
| Cardiac glycosides | 1,498 | (3%) | 5,397 | (4%) | 3,561 | (3%) |
| Nitrates | 10,250 | (21%) | 40,312 | (29%) | 29,230 | (24%) |
| Thyroid hormones | 1,208 | (2%) | 3,810 | (3%) | 3,238 | (3%) |
| Testosterone | 226 | (0%) | 731 | (1%) | 922 | (1%) |
| Fibrates | 1,997 | (4%) | 8,252 | (6%) | 6,126 | (5%) |
| Niacin | 72 | (0%) | 94 | (0%) | 67 | (0%) |
| PCSK9 inhibitors | 3 | (0%) | 3 | (0%) | 9 | (0%) |
| Cholesterol absorption | 179 | (0%) | 340 | (0%) | 379 | (0%) |
| inhibitors | | | | | | |
| Vytorin | 3 | (0%) | 1 | (0%) | 0 | (0%) |
| Bile acid sequestrants | 118 | (0%) | 156 | (0%) | 78 | (0%) |
| Omega-3 fatty acids | 28 | (0%) | 11 | (0%) | 3 | (0%) |
| Other non-statin lipid- | 1 | (0%) | 0 | (0%) | 5 | (0%) |
| modifying drugs | | | | | | |

General (before incident cardiovascular events) [median (interquartile range)]

| | | | | | | |
|---|---|---|---|---|---|---|
| Outpatient visits per year | 3.0 | (0.0-4.6) | 5.3 | (2.3-7.2) | 4.9 | (2.2-6.5) |
| Inpatient visits per year | 0.8 | (0.8-0.8) | 0.9 | (0.7-1.0) | 0.9 | (0.7-0.9)* |
| Count of medications | 5.0 | (0.0-8.0) | 7.0 | (5.0-10.0) | 7.0 | (5.0-10.0) |

PCSK9 = Proprotein convertase subtilisin/kexin type 9.
H2 = histamine type 2.
*Risk variables in the Kowloon and New Territories cohorts with no significant difference in distribution (p value ≥ 0.05) from the Hong Kong Island (Hong Kong West Cluster) under Chi-square test (categorical risk variables) or in T-test (numerical risk variables). All other risk variables were significant (p value < 0.05).

### Model Derivation

In the examples of this disclosure, 15 preselected risk variables and 8 interactive drug use options (Table 9) were identified as statistically significant and medically coherent for CVD pathogenesis. Table 9 shows adjusted hazard ratios in ML models (e.g., P-CARDIAC models) as described herein.

TABLE 9

| | | Full model | |
|---|---|---|---|
| Basic model | | (Preselected + | |
| (Preselected risk | | Supplementary risk | |
| variables) | | variables) | |
| HR (95% CI) | p value | HR (95% CI) | p value |

General

| | | | | |
|---|---|---|---|---|
| Age per year | 1.02 (1.01-1.02) | <0.0001 | 1.01 (1.01-1.01) | <0.0001 |
| Female | 0.84 (0.82-0.86) | <0.0001 | 0.86 (0.84-0.88) | <0.0001 |
| Accident and emergency visits per | 1.07 (1.06-1.08) | <0.0001 | 1.06 (1.05-1.07) | <0.0001 |
| year (prior to incident cardiovascular | | | | |
| events) | | | | |

Clinical laboratory tests

| | | | | |
|---|---|---|---|---|
| Low-density lipoprotein cholesterol | 1.06 (1.05-1.08) | <0.0001 | 1.05 (1.04-1.06) | <0.0001 |
| (mmol/L) | | | | |
| Neutrophil (10\hat{ }9/L) | 1.02 (1.02-1.03) | <0.0001 | 1.02 (1.02-1.02) | <0.0001 |
| Aspartate transaminase: alanine | 1.02 (1.02-1.03) | <0.0001 | 1.02 (1.01-1.02) | <0.0001 |
| aminotransferase ratio | | | | |

Disease and medication history

| | | | | |
|---|---|---|---|---|
| Statins | 0.84 (0.82-0.87) | <0.0001 | 0.88 (0.85-0.90) | <0.0001 |
| Hypertension | 1.16 (1.13-1.19) | <0.0001 | 1.13 (1.10-1.16) | <0.0001 |
| Diabetes | 1.38 (1.34-1.43) | <0.0001 | 1.30 (1.25-1.35) | <0.0001 |
| Atrial fibrillation | 1.09 (1.05-1.13) | <0.0001 | 1.08 (1.04-1.12) | 0.0001 |
| Myocardial infarction | 2.13 (2.06-2.21) | <0.0001 | 1.71 (1.65-1.78) | <0.0001 |
| Angina | 0.92 (0.88-0.96) | 0.0003 | 0.93 (0.89-0.97) | 0.0022 |
| Revascularization | 0.91 (0.88-0.95) | <0.0001 | 0.93 (0.90-0.96) | <0.0001 |
| Family history of diabetes | 1.37 (1.32-1.43) | <0.0001 | 1.28 (1.23-1.33) | <0.0001 |

Drug use

| | | | | |
|---|---|---|---|---|
| Antihypertensive drugs | 0.67 (0.65-0.69) | <0.0001 | 0.77 (0.74-0.79) | <0.0001 |
| Antidiabetic drugs | 0.71 (0.69-0.74) | <0.0001 | 0.77 (0.74-0.80) | <0.0001 |
| Antiplatelet drugs | 0.78 (0.75-0.80) | <0.0001 | 0.85 (0.83-0.87) | <0.0001 |
| Fibrates | 0.78 (0.73-0.84) | <0.0001 | 0.78 (0.73-0.84) | <0.0001 |
| Niacin | 0.53 (0.38-0.75) | 0.0003 | 0.56 (0.40-0.78) | 0.0007 |
| Cholesterol absorption inhibitors | 0.55 (0.49-0.63) | <0.0001 | 0.56 (0.49-0.63) | <0.0001 |
| PCSK9 inhibitors | 0.24 (0.09-0.68) | 0.0066 | 0.25 (0.09-0.69) | 0.0078 |
| Statins | 0.87 (0.85-0.90) | <0.0001 | 0.89 (0.86-0.91) | <0.0001 |
| XGBoost risk score | | | 1.03 (1.02-1.03) | <0.0001 |

Abbreviations: HR=, CI = confidence interval, PCSK9 = Proprotein convertase subtilisin/kexin type 9.

For each of the basic and full ML models, the risk variables are statistically significant (p value<0-05) when compared to those without recurrent CVD. Both models had similar estimates on the linear effects of the risk variables while the basic model's hazard ratios deviated more than 1 from the full model with a wider 95% confidence intervals (CIs), indicating more precise estimates for the full model. In some implementations, multivariate imputation with chained equations can be conducted once with <2% missing rate among the 15 mandatory risk variables. Similar hazard ratios between models reassure the consistent risk estimation across the two models.

### Model Validation

In the examples of this disclosure, validation results on the derivation cohort of P-CARDIAC full model showed satisfying discrimination and calibration performance. In various aspects, a C-statistic for an ML model has a value of at least 0.69. With reference to the example ML model, the C-statistic was 0.69, the calibration slope was 1-00, and the calibration-in-the-large was 0-03. In general, a C-statistic (also referred to as the “concordance” statistic or C-index) is a measure of goodness of fit for binary outcomes in a logistic regression model. In clinical studies, the C-statistic gives the probability a randomly selected patient who experienced an event (e.g., a disease or condition) had a higher risk score than a patient who had not experienced the event. The C-statistic is equal to the area under the Receiver Operating Characteristic (ROC) curve and ranges from 0 to 1.

A basic ML model (e.g., the P-CARDIAC basic model) showed good discrimination and calibration performance but was inferior to the full model. The C-statistic was 0.66, the calibration slope was 0.86, and the calibration-in-the-large was 0.01. The validation results are shown in FIGS. 2A and 2B, as well as Table 10.

For example, FIG. 2A illustrates a calibration plot 200 for a full ML model (e.g., full P-CARDIAC model), in accordance with various embodiments disclosed herein. As used herein, the full ML model (e.g., full P-CARDIAC model) refers to a machine learning model (e.g., ML model 502) trained on a plurality of cardiovascular risk factors (e.g., cardiovascular risk factors 504) subdivided into a first training data subset and a second training data subset, where the first training data subset comprises a preselected subset of cardiovascular risk factors (e.g., cardiovascular risk factors 504m), and where the second training subset comprises a remaining subset of cardiovascular risk factors (e.g., cardiovascular risk factors 504r). In various aspects, the preselected subset of cardiovascular risk factors comprises a set of cardiovascular risk factors for training the full ML model (e.g., full P-CARDIAC model).

For example, FIG. 2B illustrates a calibration plot 250 for a basic ML model (e.g., basic P-CARDIAC model), in accordance with various embodiments disclosed herein. As used herein, the basic ML model (e.g., basic P-CARDIAC model) refers to the machine learning model trained with the machine learning model trained on a plurality of cardiovascular risk factors (e.g., cardiovascular risk factors 504) comprising only the preselected subset of cardiovascular risk factors (e.g., cardiovascular risk factors 504m). For comparison herein, a model with only the preselected subset of cardiovascular risk factors (e.g., cardiovascular risk factors 504m) was built as a P-CARDIAC basic model.

FIGS. 2A and 2B illustrate calibration plots for a patient cohort in Hong Kong Island (Hong Kong West Cluster), which is a derivation cohort chosen, in the example embodiment, for training the ML model (e.g., ML model 502) describe herein with a 95% confidence interval. The results were measured from 10-fold cross validation. As shown in the example of FIG. 2A, the full ML model outputs a predicted 10-year risk percentage (202), which is compared against an observed 10-year risk percentage (204). Similarly, as shown for as shown in the example of FIG. 2B, the basic ML model outputs a predicted 10-year risk percentage (252), which is compared against an observed 10-year risk percentage (254). As illustrated for each of FIGS. 2A and 2B, the predicted-to-observed plot 206 of the full ML model plot 200 compared to the predicted-to-observed plot 256 of the basic ML model, demonstrates that the full ML model is more accurate with respect to user-specific cardiovascular prediction than the basic ML model. Thus, FIGS. 2A and 2B illustrate that the addition or use of the second training subset comprising the remaining subset of cardiovascular risk factors (e.g., cardiovascular risk factors 504r) increased the prediction accuracy of the ML model as described herein.

Table 10 below illustrates discrimination and calibration performance of the ML model (e.g., P-CARDIAC model) on a derivation cohort.

TABLE 10

| | Harrell's | Calibration | Calibration-in- |
|---|---|---|---|
| | C-statistic | slope | the-large |
| Basic Model | 0.66 (0.66, 0.66) | 0.86 (0.86, 0.86) | 0.01 (0.01, 0.01) |
| Full Model | 0.69 (0.69, 0.69) | 1.00 (1.00, 1.00) | 0.03 (0.03, 0.03) |

With respect to Table 10, Harrell's C-statistic is a measure of model discrimination with values ranging from 0.5 to 1 defining a probability of correct ordering for a randomly selected pair of subjects. Calibration slope is a measure of model calibration with target value of 1. Values smaller than 1 indicate overfitting, that is, values too low for low-risk patients and/or too high for high-risk patients. Values greater than 1 indicate underfitting, that is values defining too high for low-risk patients and/or too low for high-risk patients. Calibration-in-the-large is a measure of model calibration with a target value of 0. Values greater than 0 means a given ML model overestimates risk in general. Values smaller than 0 means a given ML model underestimates risk in general. With respect to the present disclosure herein, results were measured from 100 repeats of 10-fold cross validation.

FIG. 3 illustrates calibration plots (e.g., plots 302-308) for various models as validated on validation data, in accordance with various embodiments disclosed herein. For example, with respect to plots 302 and 304, FIG. 3 illustrates an ML model (e.g., ML model 502), such as full and basic ML models, validated on validation data, respectively. For example, plots 302 and 304 of FIG. 3 provides an example of validating the ML model trained by with derivation data (e.g., Hong Kong Island (Hong Kong West Cluster) derivation data) as described for FIGS. 2A and 2B with validation data comprising data from different cohorts (e.g., data of cohorts of Kowloon and New Territories). More generally, FIG. 3 illustrates plots for models for different validation cohorts of data (e.g., data of cohorts of Kowloon and New Territories). In particular, plots 302 correspond to a full ML model validated against each of the Kowloon and New Territories data cohorts, respectively. Plots 304 correspond to a basic ML model validated against each of the Kowloon and New Territories data cohorts, respectively. Plots 306 correspond to a SMART2 model validated against each of the Kowloon and New Territories data cohorts, respectively. Finally, Plots 308 correspond to a TRS-2 model validated against each of the Kowloon and New Territories data cohorts, respectively.

In the example of FIG. 3, calibration plots (e.g., plots 302-308) for validating the various models on the validation cohort of data have an error bar with a 95% confidence interval. P-values of plots 308 were Mann-Kendall tested for significance of monotonic trend. The P-value larger than 0.05 indicates no significant sign of increasing or decreasing trend in observed risk when predicted risk score increases. The result of the full ML model (e.g., the full P-CARDIAC) is validated on New Territories cohort (lower left) was after recalibration as described herein for FIG. 6.

As shown for FIG. 3, validation of the P-CARDIAC full model (e.g., plots 302) across validation cohorts demonstrate accurate discrimination and calibration performance, especially when compared to the remaining models of the remaining plots 304-308. For the full model, the C-statistic for the Kowloon and New Territories cohorts were 0.62 and 0.64, the calibration slope was 0.75 and 0.93, and the calibration-in-the-large was 0.04 and 0.01, respectively. By comparison, the P-CARDIAC basic model (e.g., plots 304) showed accurate discrimination and calibration performance but was inferior to the full model. For the basic model, the C-statistic for Kowloon and New Territories cohorts were 0.60 and 0.62, the calibration slope was 0.66 and 0.75, and the calibration-in-the-large was 0-01 and 0-03, respectively. As a benchmark comparison, validation of both TRS-2° P and SMART2 risk scores (e.g., plots 306 and 308, respectively) underperformed regarding discrimination and risk stratification performance when compared to each of the full and basic ML models (e.g., plots 302 and 304, respectively). In particular, the C-statistic was lower than 0.55 for both validation cohorts TRS-2° P and SMART2 risk scores (e.g., plots 306 and 308, respectively).

The validation results of FIG. 3 are further summarized in Tables 11-13.

TABLE 11
Mean (95% Confidence Interval) of Harrell's C-statistic on validation cohorts

| | P-CARDIAC (full) | P-CARDIAC (basic) | SMART2 | TRS-2° P |
|---|---|---|---|---|
| Kowloon | 0.62 (0.62, 0.62) | 0.60 (0.60, 0.60) | 0.55 (0.55, 0.55) | 0.53 (0.53, 0.53) |
| New Territories | 0.64 (0.64, 0.64) | 0.62 (0.62, 0.62) | 0.55 (0.55, 0.55) | 0.54 (0.54, 0.54) |

In Table 11, a measure of model discrimination with values ranging from 0.5 to 1 defines a probability of correct ordering for a randomly selected pair of subjects. Values were measured from 1000 bootstrap replicates.

TABLE 12
Mean (95% Confidence Interval) of calibration
slope on validation cohorts

| | P-CARDIAC | P-CARDIAC | |
|---|---|---|---|
| | (full) | (basic) | SMART2 |
| Kowloon | 0.75 (0.74, 0.75) | 0.66 (0.66, 0.66) | 0.38 (0.38, 0.38) |
| New Territories | 0.93 (0.93, 0.93) | 0.75 (0.75, 0.75) | 0.39 (0.39, 0.39) |

In Table 12 shows a measure of model calibration with a target value of 1. Values smaller than 1 indicate overfitting defining too low for low-risk patients and/or too high for high-risk patients. Values greater than 1 indicate underfitting defining too high for low-risk patients and/or too low for high-risk patients. Values were measured from 1000 bootstrap replicates.

TABLE 13
Mean (95% Confidence Interval) of calibration-
in-the-large on validation cohorts

| | P-CARDIAC | P-CARDIAC | |
|---|---|---|---|
| | (full) | (basic) | SMART2 |
| Kowloon | 0.04 (0.04, 0.04) | 0.01 (0.01, 0.01) | 0.10 (0.10, 0.10) |
| New Territories | 0.01 (0.01, 0.01) | 0.03 (0.03, 0.03) | 0.11 (0.11, 0.11) |

In Table 13, a measure of model calibration with target value of 0. Values greater than 0 means the model overestimates risk in general. Values smaller than 0 means the model underestimates risk in general. Values were measured from 1000 bootstrap replicates.

In summary, with respect to the validation data demonstrated for FIG. 3 and Tables 11-13, the ML models (P-CARDIAC models), i.e., both full and basic ML models, showed improved performance on the three derivation and validation cohorts. The full model had better performance than the basic model as it accurately accounted for the nonlinear effects and the effects from supplementary risk variables (504r). On the other hand, TRS-2° P and SMART2 underperformed when adapted to the two cohorts for the example Chinese populations.

### Clinical Utility

In the examples of this disclosure, decision curve analysis of the two validation cohorts was similar to the results of FIG. 3, as demonstrated for FIG. 4. In particular, FIG. 4 illustrates decision curves of the various models of FIG. 3 with their respective net benefit comparisons, in accordance with various embodiments disclosed herein. FIG. 4 shows a net benefit (y-axis) across a threshold probability percentage (x-axis) for each of the validation cohorts, with plot 402 illustrated for the Kowloon cohort and plot 404 illustrated for the New Territories cohort. The threshold probability (x-axis) was the predicted 10-year cardiovascular disease recurrence risk.

As illustrated for FIG. 4, a full ML model (e.g., the P-CARDIAC full model) performed better (e.g., had more accurate predictions or net benefits with respect to prediction accuracy) than the basic ML model (e.g., the P-CARDIAC basic model). Both P-CARDIAC models had similar and greater net benefits across a larger range of threshold risks compared with the “treat all” strategy (e.g., treating all patients), TRS-2° P, and SMART2 models or otherwise risk scores. The ML models described herein (e.g., the P-CARDIAC model) demonstrated clinical values for decision-making when the threshold risk was under 90%.

### Graphic User Interface (GUI) Design

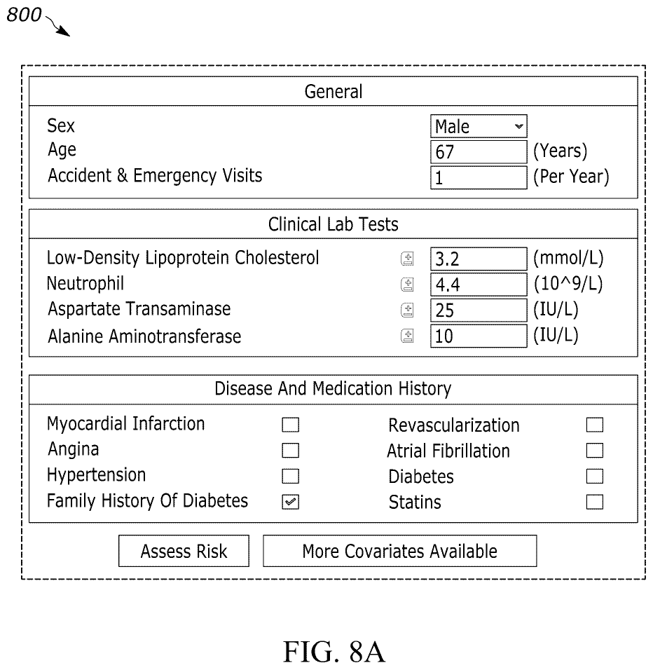

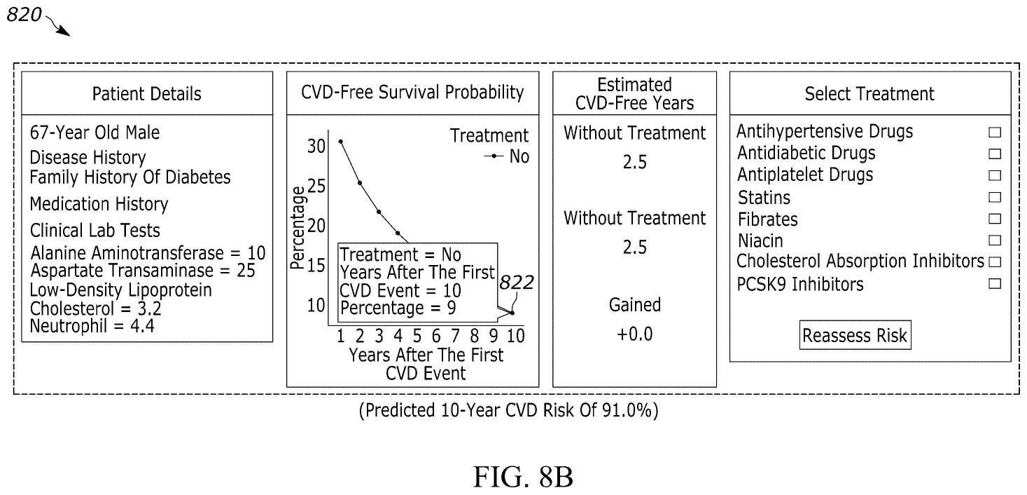

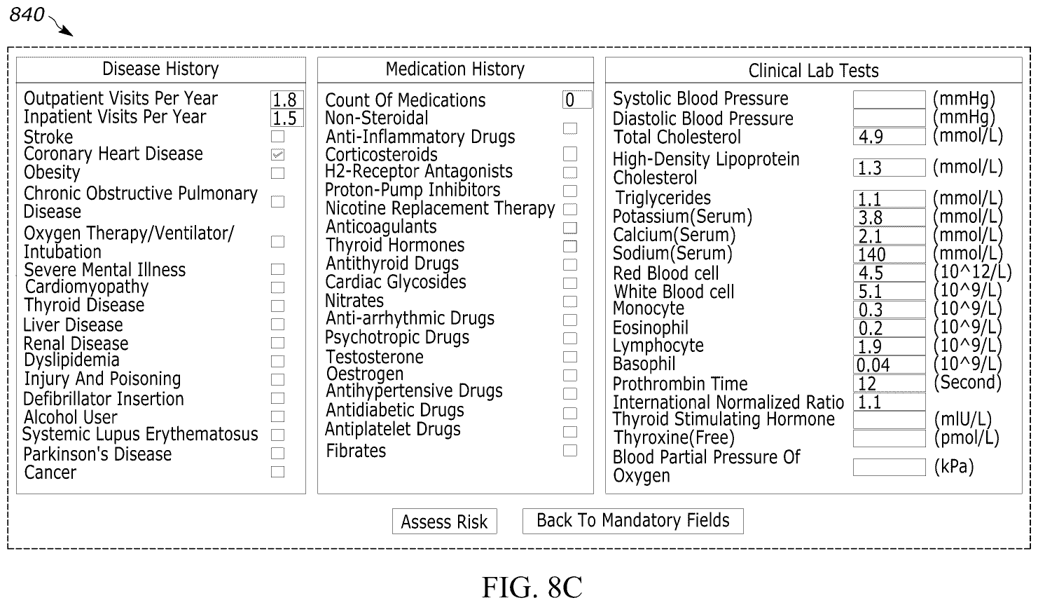

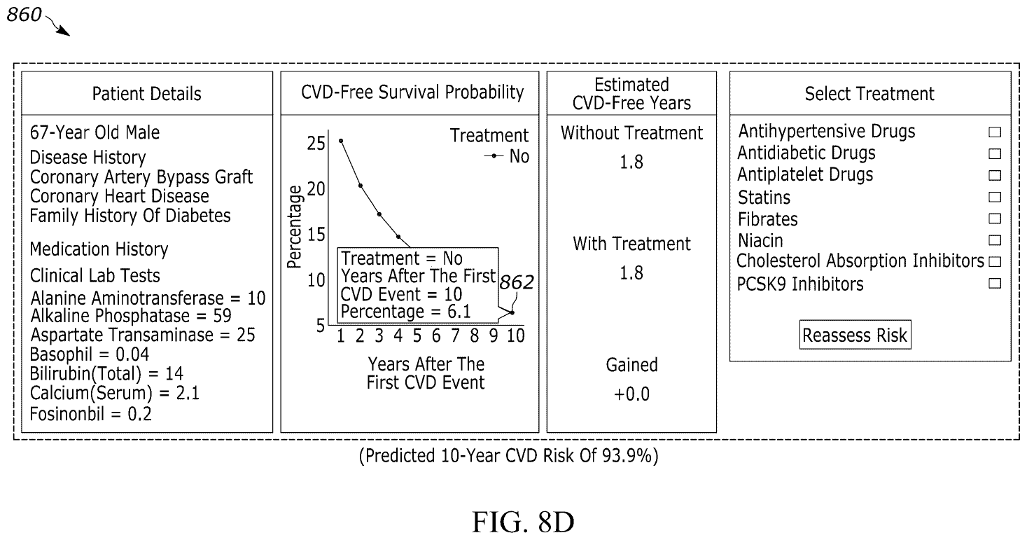

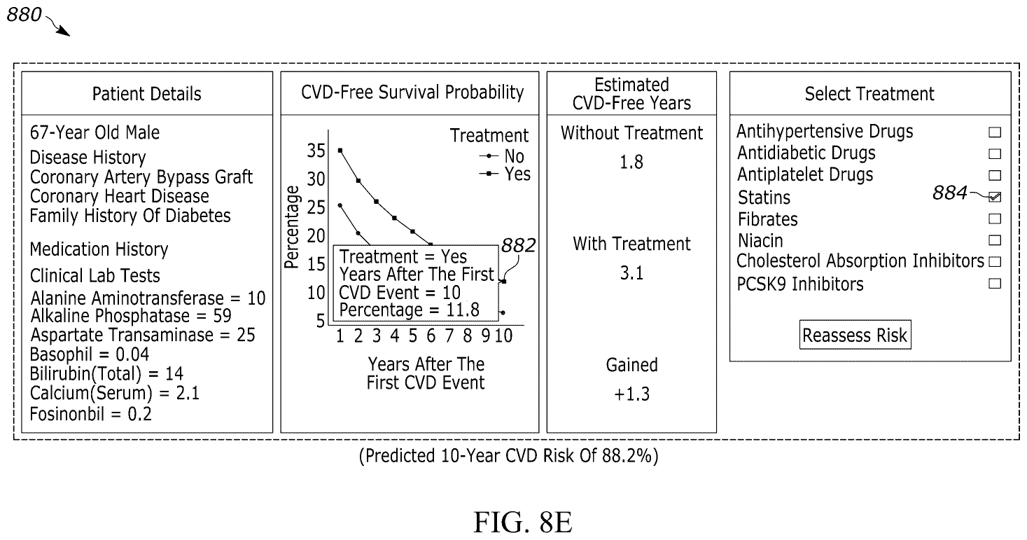

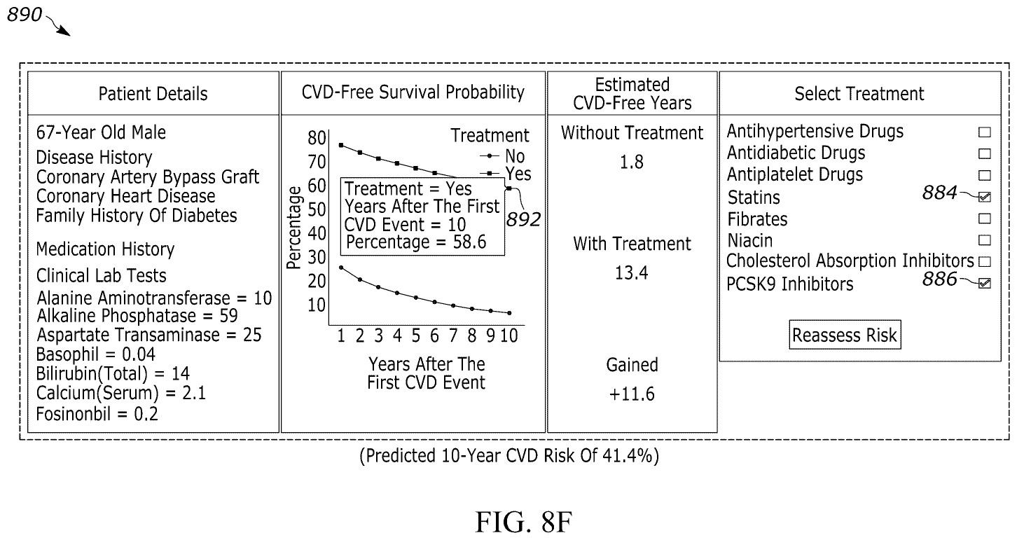

FIGS. 8A-8F illustrates a series of graphical user interfaces (GUIs) 800-890 for accessing or otherwise interacting with an ML model (e.g., ML model 502) as described herein. The GUIs may be used, for example, to receive cardiovascular data specific to a given user (user-specific data). That is, in various aspects, a GUI may be configured to receive the user-specific cardiovascular data of the user, and wherein the GUI is further configured to provide the user-specific cardiovascular data as input to the ML model. For example, users of the GUIs (e.g., a nurse, doctor, or other medical professional) can input up to risk variables (504m) in fields representing preselected values for a quick evaluation of CVD risk (FIG. 8A). In addition, additional risk variables (504r) can be further inputted in the supplementary field (see, e.g., FIG. 8C) for a more comprehensive evaluation. The more risk variables (504r) submitted in the supplementary field, the more accurate the prediction can be (see, e.g., FIGS. 8C and 8D).

A GUI may also be used for displaying a user-specific cardiovascular prediction of the user as determined and output by an ML model (e.g., ML model 502). In various aspects, the user-specific cardiovascular prediction may comprise a cardiovascular risk score of the user that defines the user's risk of a cardiovascular event within a given time period (e.g., a 10-year time period). In some aspects, a user-specific cardiovascular prediction comprises a user-specific medical prescription predicted to reduce the user's CVD risk. Such user-specific medical prescription may comprise, by way of non-limiting example, a medical prescription for any one or more of antihypertensive drugs, antidiabetic drugs, antiplatelet drugs, statins, fibrates, niacin, PCSK9 inhibitors, cholesterol absorption inhibitors, and/or other drugs or otherwise treatments as described herein. Additionally, or alternatively, a user-specific cardiovascular prediction causes generation of a user-specific activity predicted to reduce the user's CVD risk. Such user-specific activity may comprise a recommendation or information regarding more healthcare examinations or increased exercise.

Furthermore, in some aspects, drug use risk variables were designed as interactive selection options, where various types (e.g., 8 types) of drug classes can be selected for evaluation of potential synergetic treatment effects to guide possible treatment plans (e.g., see FIGS. 8E and 8F). In such aspects, an ML model is further trained with data of one or more drug classes identified for reducing cardiovascular disease (CVD), including, by way of non-limiting example, one or more drug classes or subclasses as shown herein for Tables 4 or 5. Further user-specific cardiovascular data of the user may be provided to the ML model, where such user-specific data comprises a selection of one or more of the drug classes. The ML model may then output a user-specific cardiovascular prediction of the user that comprises a cardiovascular disease (CVD) risk prediction that predicts the user's cardiovascular after using the one or more of the drug classes as selected.

More specifically, FIG. 8A illustrates a graphical user interface 800 depicting fields for receiving user-specific data corresponding to a preselected subset of cardiovascular risk factors (504m), in accordance with various embodiments disclosed herein. For example, such cardiovascular risk factors (504m), and/or those shown for Table 7, may comprise data corresponding to preselected covariates (e.g., preselected covariates 502pscv). Additionally, or alternatively, as shown for FIG. 8A, additional and/or other such fields and related data may be used or submitted including, for example general fields (e.g., sex, age, and accident and emergency visit field(s); clinical lab test fields (e.g., low-density lipoprotein cholesterol, neutrophil, aspartate, transaminase, and alanine aminotransferase field(s)); and/or disease and medication history fields (e.g., myocardial infarction, angina, hypertension, family history of diabetes, revascularization, atrial fibrillation, diabetes, and statin field(s)). It is to be understood that additional, fewer, or different fields may be used or implemented for the preselected fields of FIG. 8A, including fields for all such preselected values or covariates as described herein.

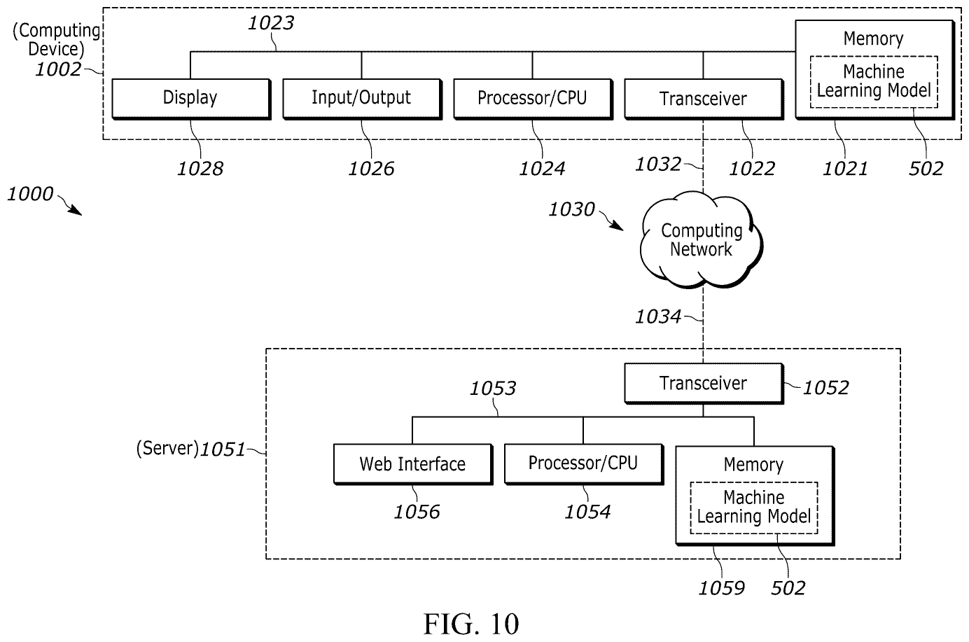

FIG. 8B illustrates a graphical user interface 820 depicting output of an ML model (e.g., ML model 502) after inputting the values of the preselected subset of cardiovascular risk factors (504m) of the GUI of FIG. 8A, in accordance with various embodiments disclosed herein. The ML model that has provided output may comprise ML model 502 as described herein. The ML model may be stored in a memory of a client device or sever, for example, as described herein for FIG. 10. In the example of FIG. 8B, user-specific details have been provided for or by a user. Such data indicates that the user (patient) is a 67-year-old male, with a family history of diabetes, no medication history, and clinical lab tests for, as example, alanine aminotransferase (value of 10), aspartate transaminase (value of 25), low-density lipoprotein cholesterol (value of 3.2), and neutrophil (value of 4.4). As shown for FIG. 8B, the ML model 502 outputs a user-specific cardiovascular prediction of the user. The user-specific cardiovascular prediction of the user further comprises CVD-free survival probability graphically depicted as a curve on a plot of a percentage likelihood (y-axis) of contracting CVD over a 10-year period (x-axis). The user-specific cardiovascular prediction further comprises a cardiovascular risk score (822) of the user, where the risk score predicts a 91% chance of CVD risk for the user within a 10-year period. The specific cardiovascular prediction further indicates estimated CVD-free years with and without treatment (2.5 and 2.5 years, respectively) for the user. In the example of FIG. 8B, no treatment is selected so the number of years gained is +0.0.

FIG. 8C illustrates a graphical user interface 840 depicting fields for receiving user-specific data corresponding to a remaining subset of cardiovascular risk factors (504r), in accordance with various embodiments disclosed herein. For example, such cardiovascular risk factors may comprise those corresponding to remaining subset of cardiovascular risk factors (504r), and/or those shown for Table 8, as described herein. Additionally, or alternatively, as shown for FIG. 8C other such fields and related data may be compared including, for example disease history fields, medication history fields, and clinical lab test fields, each as depicted for FIG. 8C. In the example of FIG. 8C, the input is received in addition (supplementary) to the values received from the GUI of FIG. 8A. It is to be understood that additional, fewer, or different fields may be implemented for the remaining (or supplementary) fields of FIG. 8C, including fields for all such remaining (or supplementary) values as described herein.

FIG. 8D illustrates a graphical user interface 860 depicting output of the ML model (e.g., ML model 502) after additionally inputting the values of remaining subset of cardiovascular risk factors (504r), in accordance with various embodiments disclosed herein. The ML model may comprise ML model 502 as described herein. In the example of FIG. 8D, user-specific details have been provided for a user (e.g., the user as described for FIG. 8B). Such data indicates that the user (patient) is a 67-year-old male, with a family history of diabetes, no medication history, and clinical lab tests for, as example, alanine aminotransferase (value of 10), aspartate transaminase (value of 25), low-density lipoprotein cholesterol (value of 3.2), and neutrophil (value of 4.4). The data is updated based on the supplementary (remaining) information provided via GUI 840 as shown for FIG. 8C. This includes coronary artery bypass graft data, coronary heart disease history, and additional clinical lab test information. In the example of FIG. 8D, ML model 502 outputs a user-specific cardiovascular prediction of the user. The user-specific cardiovascular prediction of the user further comprises CVD-free survival probability graphically depicted as a curve on a plot of a percentage likelihood (y-axis) of the user contracting CVD over a 10-year period (x-axis). The user-specific cardiovascular prediction further comprises a cardiovascular risk score (862) for the user, where the risk score predicts a 93.9% chance of CVD risk of the user within a 10-year period. The GUI 860 demonstrates updated ML model output when the ML model 502 is provided with additional user-specific data (e.g., remaining data (504r)). As shown for FIG. 8D, the updated output comprises an increased risk of CVD for the specific user, where the specific cardiovascular prediction indicates estimated CVD-free years of the user with and without treatment (1.8 and 1.8 years, respectively). In the example of FIG. 8D, no treatment is selected so the number of years gained is +0.0.

FIG. 8E illustrates a graphical user interface 880 depicting output of the ML model (e.g., ML model 502) after inputting or otherwise providing a selection of one or more of the drug classes (e.g., one or more drug classes or subclasses as shown herein for Tables 4 or 5), in accordance with various embodiments disclosed herein. In the example of FIG. 8E, user-specific details have been provided for a user (e.g., the user as described for FIGS. 8A-8D). In the example of FIG. 8D, a treatment option 884 has been made from the GUI that indicates a statins drug indication is added to the input of the ML model. The treatment option is provided to ML model 502 to update the user-specific cardiovascular prediction for the user, where the user is assumed to take a statins drug. In the example of FIG. 8E, ML model 502 outputs a user-specific cardiovascular prediction of the user comprising a CVD-free survival probability graphically depicted as a curve on a plot of percentage likelihood (y-axis) of the suer contracting CVD over a 10-year period (x-axis). The user-specific cardiovascular prediction further comprises a cardiovascular risk score (882) for the user, where the risk score predicts an 88.2% chance of CVD risk of the user within a 10-year period. The GUI 880 demonstrates updated ML model output when the ML model 502 is provided with a treatment option of a drug selected for a specific drug class. As shown for FIG. 8E, the updated output comprises a decreased risk of CVD for the specific user, where the specific cardiovascular prediction indicates estimated CVD-free years of the user with and without treatment (1.8 and 3.1 years, respectively). In the example of FIG. 8D, the selected treatment option (e.g., for the user to take statins) is expected to increase the user's CVD-free years by +1.3 years.

FIG. 8F illustrates a graphical user interface depicting output of the ML model (e.g., ML model 502) after inputting or otherwise providing a second selection of one or more of the drug classes (e.g., one or more drug classes or subclasses as shown herein for Tables 4 or 5), in accordance with various embodiments disclosed herein. In the example of FIG. 8F, user-specific details have been provided for a user (e.g., the user as described for FIGS. 8A-8E). In the example of FIG. 8F, a second treatment option 886 has been made from the GUI indicating a PCSK9 inhibitor selection, where it is assumed the user will take a PCSK9 inhibitor drug. The treatment option is provided to ML model 502 to update the user-specific cardiovascular prediction for the user. In the example of FIG. 8F, ML model 502 outputs a user-specific cardiovascular prediction of the user comprising a CVD-free survival probability graphically depicted as a curve on a plot of percentage likelihood (y-axis) of the user contracting CVD over a 10-year period (x-axis). The user-specific cardiovascular prediction further comprises a cardiovascular risk score (892) for the user, where the risk score predicts an 41.4% chance of CVD risk within a 10-year period. The GUI 890 demonstrates updated ML model output when the ML model 502 is provided with a second treatment option of a drug selected for a specific drug class. As shown for FIG. 8F, the updated output comprises a decreased risk of CVD for the specific user, where the specific cardiovascular prediction indicates estimated CVD-free years for the user with and without treatment (1.8 and 13.4 years, respectively). In the example of FIG. 8F, the selected treatment option (e.g., for the user to take additionally take a PCSK9 inhibitor with statins) is expected to increase the user's CVD-free years by +11.6 years.

It is to be understood that FIGS. 8A-8F illustrate example graphical user interfaces as rendered on a display (e.g., display 1028) of a computing device (e.g., computing device 1002 as shown for FIG. 10) in accordance with various aspects disclosed herein. For example, as shown in FIGS. 8A-8F, the graphical user interfaces 800-890 may be implemented or rendered via a web browser, such as via a web browser application, e.g., SAFARI and/or GOOGLE CHROME web browsers, app(s), or other such web browsers or the like, for accessing a web interface (e.g., web interface 1056 as described for FIG. 10).

In other aspects, graphical user interfaces 800-890 may be implemented or rendered via an application (app) executing on user computing device (e.g., computing device 1002). For example, graphical user interfaces 800-890 may be implemented or rendered via a native app executing on user computing device 1002 as described for FIG. 10. In such aspects, the user computing device 10002 may comprise a mobile device such as an iOS-enabled the APPLE iOS operating system and/or an ANDROID enabled device implementing the ANDROID operating system. A user may use the computing device to execute one or more native applications (apps) on its operating system, including, for example, an app that would generate graphical user interfaces as described herein for FIGS. 8A-8F. Such native apps may be implemented or coded (e.g., as computing instructions) in a computing language (e.g., SWIFT) executable by the user computing device operating system (e.g., APPLE iOS) by the processor (e.g., processor 1024) of user computing device. In such aspects, the app may communicate with a server (e.g., server 1051) for transmission of receipt of data and information, such as training data or input/output data or information (e.g., risk scores or other information describe herein) for display (e.g., via display 1028 as described for FIG. 10).

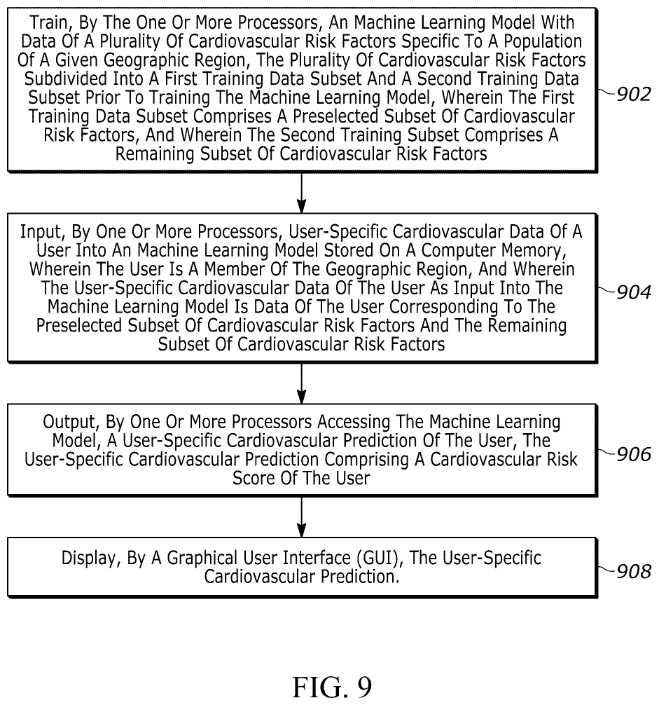

FIG. 9 illustrates a machine learning (ML)-based method 900, or otherwise algorithm, for predicting cardiovascular disease, in accordance with various embodiments disclosed herein. At block 902, method 900 comprises training, by the one or more processors (e.g., processors 1024 and/or 1054), an ML model (e.g., ML model 502) with data of a plurality of cardiovascular risk factors (e.g., plurality of cardiovascular risk factors 504). In various aspects, the data of the plurality of cardiovascular risk factors may be specific to a population of a given geographic region (e.g., one or more regions of China). In some aspects, the ML model may comprise a Cox proportional hazards model. Additionally, in some aspects, the one or more processors may implement or apply a gradient boosting algorithm (e.g., the XGBoost algorithm) to the second training subset of the remaining subset of cardiovascular risk factors (504r) to enhance the Cox proportional hazards model. In additional aspects, the geographic region defining the plurality of cardiovascular risk factors on which the ML model is trained comprises a plurality subregions or cohorts (e.g., cohort 130 and/or cohort 160, as shown and described for FIG. 1) comprising individuals located within each respective subregion or cohort. In some aspects, a C-statistic for the ML model has a value of at least 0.69.

With further reference to block 902, the plurality of cardiovascular risk factors is subdivided into a first training data subset and a second training data subset prior to training the ML model. The first training data subset comprises a preselected subset of cardiovascular risk factors (e.g., preselected subset of cardiovascular risk factors 504m). In some aspects, the preselected subset of cardiovascular risk factors (504m) comprises risk factors selected from one or more risk categories defining indications of cardiovascular health. For example, the one or more risk categories may comprise demographic factors, family history of disease, healthcare utilization, clinical laboratory testing, medication history, disease history, and drug use, or other such factors as described herein. Preselected risk factors that may be used for training ML model 502 are also illustrated by Table 7 herein. At least in some aspects, at least a portion of the preselected subset of cardiovascular risk factors (504m) comprises imputed data generated to replace missing values. In some embodiments, the preselected risk factors (e.g., the preselected subset of cardiovascular risk factors 504m) are determined based on one or more selection criteria. For example, the one or more selection criteria can comprise at least one of p-value, data completeness, event rate, and medical relatedness. In some embodiments, the preselected risk factors are determined by ranking the plurality of risk factors (e.g., the plurality of cardiovascular risk factors 504) and selecting the top one or more risk factors. In this situation, ranking the plurality of risk factors can be based on one or more hazard ratios.

The second training subset comprises a remaining subset of cardiovascular risk factors (e.g., a remaining subset of cardiovascular risk factors 504r). In some embodiments, the remaining subset of risk factors (e.g., remaining subset of cardiovascular risk factors 504r) are determined by ranking the plurality of risk factors (e.g., the plurality of cardiovascular risk factors 504) and selecting the remaining one or more risk factors other than the preselected subset of risk factors. In this situation, one or more statistical and/or mathematical techniques (e.g., gradient boosting algorithm) can be applied to the data of the remaining subset of risk factors to generate an additional covariate for use in the ML Model. Such additional covariate can be used to account for a nonlinear relationship between the remaining subset of risk factors and the preselected subset of risk factors. In some embodiments, such additional covariate can be considered as a calculated risk factor in addition to the preselected subset of risk factors that can be used in the ML Model. Remaining or otherwise supplementary risk factors that may be used for training ML model 502 are also illustrated by Table 8 herein. In various aspects, the remaining subset of cardiovascular risk factors (504r) are not imputed. Non-imputed data may comprise raw data that may contain missing on incomplete values. Use of non-imputed data allows the disclosed ML-based systems and methods herein to have a reduced memory data storage requirement, while still allowing the ML model to be highly predictive.

Still further, in some aspects, ML model (e.g., ML model 502) is further trained with data defining one or more threshold risks, where each threshold risk defines a magnitude of a clinical health benefit (e.g., +11.4 years without a CVD occurrence as demonstrated for FIG. 8F) to a user of the geographic region (e.g., China).

At block 904, method 900 further comprises inputting, by one or more processors, user-specific cardiovascular data of a user into an ML model stored on a computer memory. The user may comprise a member of the geographic region (e.g., China). The user-specific cardiovascular data of the user as input into the ML model is data of the user corresponding to the preselected subset of cardiovascular risk factors (504m) and the remaining subset of cardiovascular risk factors (504r). In some aspects, a graphical user interface (GUI) is configured to receive the user-specific cardiovascular data of the user. In such aspects, the GUI may be further configured to provide the user-specific cardiovascular data as input to the ML model.

At block 906, method 900 further comprises outputting, by one or more processors accessing the ML model, a user-specific cardiovascular prediction of the user, for example, as described with respect to FIGS. 8A-8F. In some aspects, the user-specific cardiovascular prediction is a cardiovascular disease (CVD) risk prediction for the user in a 10-year timeframe. The user-specific cardiovascular prediction may comprise a cardiovascular risk score of the user (e.g., a percent chance of the user experiencing a CVD occurrence within a 10-year period). It should be understood, however, that a 10-year period or otherwise timeframe is but one example. Different and/or additional time periods or otherwise timeframes may be implemented, which may include, by way of non-limiting example, any one or more of a 5-year period, a 15-year period, a 20-year period, and so on, e.g., for specific stages of the user's lifespan.

In additional aspects, the ML model is further trained on data of one or more drug classes identified for reducing cardiovascular disease (CVD), for example, as shown for FIGS. 8E and 8F. In such aspects, the user-specific cardiovascular data of the user as input into the ML model further comprises a selection of one or more of the drug classes. The user-specific cardiovascular prediction of the user comprises a cardiovascular disease (CVD) risk prediction that predicts the user's cardiovascular after using the one or more of the drug classes as selected.

At block 908, method 900 further comprises displaying, by a graphical user interface (GUI), the user-specific cardiovascular prediction. In additional aspects, the GUI may provide graphical fields or selections for selecting one or more types of drug classes for selection or generation of a user-specific plan to address the user's cardiovascular health, for example, as shown for FIGS. 8E and 8F. In additional aspects, the user-specific cardiovascular prediction comprises a user-specific medical prescription predicted to reduce the user's CVD risk. In still further aspects, the user-specific cardiovascular prediction comprises causes generation of a user-specific activity predicted to reduce the user's CVD risk. Such aspects may include, for example, more frequent health care visits, additional medical exams, or increased exercise.

While FIG. 9 describes a method specific to cardiovascular diseases, the machine learning (ML)-based systems and methods may be applied to other disease data to have a same predictive output as described herein. For example, in one aspect, a machine learning (ML)-based method for predicting disease may comprise training, by the one or more processors, an ML model with data of a plurality of disease risk factors, which may be (but need not specifically be) specific to a population of a given geographic region (e.g., China). The disease may relate to a different disease (e.g., a kidney disease, diabetes, high blood pressure). In such aspects, the plurality of disease risk factors is subdivided into a first training data subset and a second training data subset prior to training the ML model. The first training data subset may comprise a preselected subset of disease risk factors and a second training subset comprises a remaining subset of disease risk factors. The disease risk factors may be specific to the given disease. Such method may further comprise inputting, by one or more processors, user-specific health data of a user into an ML model stored on a computer memory. The user may be a member of the geographic region. Further, the user-specific health data of the user as input into the ML model may comprise data of the user corresponding to the preselected subset of disease risk factors and the remaining subset of disease risk factors. The method may further comprise outputting, by one or more processors accessing the ML model, a user-specific disease prediction of the user, the user-specific disease prediction comprising a disease risk score of the user. Still further, the method may comprise displaying, by a graphical user interface (GUI), the user-specific disease prediction. The disclosure herein with respect to cardiovascular disease data, factors, training, predictions, and otherwise applies here to the ML-based method for predicting disease in general.

FIG. 10 illustrates a machine learning (ML)-based system or platform 1000 configured to predict cardiovascular disease, in accordance with various embodiments disclosed herein. For example, the ML-based system or platform 1000 can implement ML-based method 900 or otherwise algorithm as described herein for FIG. 9. With respect to FIG. 10, a computing device 1002 is configured for training, implementing, and/or display output of an ML model configured to predict cardiovascular disease as described herein (e.g., for FIG. 5). Computing device 1002 may comprise processor 1024 for executing computing instructions, for training and/or accessing an ML-model, or otherwise implementing the methods or otherwise algorithms as described herein. In various aspects, the computing instructions may comprise instructions in one or more programming languages including, by way of non-limiting example, the Python programming language, Java, C, C++, C# or the like. Processor 1024 may be a microprocessor, or central processing unit (CPU) such as an INTEL®-based, AMD®-based, or other such microprocessor. Processor 1024 may be responsible for the control of the various components communicatively coupled via bus 1023. For example, processor 1024 may control storage of training data, such as data of the plurality of cardiovascular risk factors (e.g., cardiovascular risk factors 504 or subsets thereof as shown and described for FIGS. 1 and 5), which may be specific to a population of given geographic region, which, in one embodiment, can be stored in memory 1021. The ML-Model, once trained by processor 1024, may also be stored in memory 1021.

In addition, processor 1024 may receive commands or other instructions from input/output component 1026. Input/output component 1026 may be interfaced with, or otherwise connected to, various input/output devices, such as keyboard, mouse, or similar components. Such components may be used to access or otherwise manipulate or data of the plurality of cardiovascular risk factors (e.g., cardiovascular risk factors 504) (e.g., in memory 1021) or risk factors for given users as output by a trained ML model as described herein. Processor 1024 may also be communicatively connected to display 1028. Display 1028 may be a display screen, where processor 1024 would render or display user-specific cardiovascular prediction(s) or other data or information, as described herein (for example as shown for any one or more of FIGS. 8A-8F), on the display screen of display 1028.

Processor 1024 may further be communicatively connected, via bus 1023, to transceiver 1022. Processor 1024, via transceiver 1022, may be communicatively coupled over computing network 1030 (e.g., the Internet) to server 1051. In the embodiment of FIG. 10, server 1051 may send 1034 and receive 1032 data (e.g., such as training data or user-specific cardiovascular prediction) via computer network 1030, via a processor 1054 and transceiver 1052 of server 1051. Server 1051 may comprise a computer server or cloud platform, such as MICROSOFT AZURE, GOOGLE CLOUD, AMAZON AWS, or the like. Server 1051 may comprise a memory 1059, communicatively coupled to processor 1054 via bus 1053, for storing data, computing instructions, the ML-model 502, and/or other data or information (e.g., training data) as described herein. Still further, server 1051 provides a web interface 1056, such as described and demonstrated herein for FIGS. 8A-8E. The web interface 1056 may be implemented via client-server frameworks such as MICROSOFT ASP, RUBY ON RAILS, or other such client-server technologies for generating web pages and screens as depicted for FIGS. 8A-8E herein.

In the embodiment of FIG. 10, memory 1021 and/or memory 1059 may store program instructions to cause either one or both of processors 1024 and/or 1054 to execute the program instructions to implement machine learning (ML)-based method or otherwise algorithm for predicting cardiovascular disease as described herein (e.g., for FIG. 5). In various aspects the memory 1021 and/or memory 1059 may comprise tangible, non-transitory computer-readable medium for storing the program instructions. The program instructions may be program code in a programming language such as Python, Java, C#, or other programming language. In some embodiments, the program instructions may be client-server based, where communicates to processor 1054 of server 1051 over computing network 1030. In such embodiments, remote processor 1024 may request data, such as training data (e.g., data of the plurality of cardiovascular risk factors (504) as stored in memory 1059). Such data may be requested by processor 1024 via an online application programming interface (API) (e.g., stored in memory 1059 of sever 1051), where the API comprises, by way of non-limiting example, a representational state transfer (RESTful) API, where processor 1024 accesses the API to receive data or information from remote processor 1054 via computer network 130. In additional aspects, the ML model 502 may be stored in the memory 1059 of server 1051, where processor 1024 of client device accesses ML model 502 remotely, via computer network 1030 via an API exposed by server 1051, to provide input of user-specific data and/or information (e.g., the user-specific cardiovascular prediction comprising a cardiovascular risk score of the user). For example, in various aspects, web interface 1056 can provide server code or an application API, executing by processor 1054 of server 1051, for computing device 1002 to access server 1051, including accessing ML model 502 stored in memory 1059, where server 501 can host ML model 502, and its output can be provided to computing device 1002 via computer network 1030 when computing device 1002 requests ML model 502 to output a user-specific cardiovascular prediction as described herein.

### Aspects of the Disclosure

The ML model as described herein provides a novel technology for predicting recurrent CVD events, which may be in a given geographic region or population (e.g., a Chinese geographic region or population), and which may use cohorts of data from the given geographic region or population, for example, as described herein for FIG. 1. The ML model as described herein (e.g., ML model 502 or otherwise the P-CARDIAC model) demonstrates reliable performance of recurrent CVD risk prediction in 10 years on three derivation and validation cohorts. The ML models described herein (e.g., ML model 502 or otherwise the P-CARDIAC model) have better performance in risk prediction than existing CVD risk scores such as TRS-2° P and SMART2. The results described herein also results also the full ML model has superior performance to the basic ML model.

As described herein, in some aspects, information or data of various drug classes or subclasses (e.g., as described herein for Tables 4 and 5) were used to train the ML model (e.g., ML model 502) as interactive covariates for the model to evaluate such drug or drug classes bias-mitigated, risk stratified, and geographic region-specific (e.g., China region) treatment effects. Among the drug classes and/or subclasses included in the interactive covariates, classes had hazard ratios lower than 1 whilst PSCK9 inhibitors had the lowest. This observation indicates that drug treatment with indications for risk variable CVD such as lipid-modifying drugs, antihypertensive, and antidiabetic drugs all have a beneficial effect on reducing CVD risk. The ML model (e.g., ML model 502) described herein also considers, and is trained to on, prior statin use for primary prevention prior to the first CVD event. Patients who received statins as primary prevention prior to the first CVD event were identified by the ML model as having a lower risk of recurrent CVD events, independent of whether such patients (users) continued statin therapy.

As described herein, in some aspects, an ML model (e.g., ML model 502), such as the P-CARDIAC model, can be developed using hybrid statistical-machine learning algorithms, which is novel in the field of CVD risk prediction. By contrast, traditional prediction tools rely on linear combinations of a selected pool of small number of covariates, which are easily interpreted, but do not consider the massive nonlinear effects and often lack accuracy. On the other hand, in recent years many ML and deep learning methods have emerged that takes into consideration the complex relationships of all massive covariates to yield high accuracy. However, since these models lack linear representations of the covariates, the effects of the risk variables are uncertain and unclear. Therefore, the ML approach is described as the “black box approach”. The ML model, and related systems and methods described herein, is an improvement over traditional approaches by implementing selection of a pool of clinically relevant covariates using statistical methods (e.g., see FIGS. 1 and 5), then using ML (e.g., gradient boosting) for a remainder of a large number of covariates and their complex effects. As described herein XGBoost may be used as an ML method for gradient boosting. This novel hybrid method showed significantly better performance than the traditional statistical method by comprehensively considering a large pool of covariates, including commonly known risk factors, such as blood pressure, hemoglobin A1c, blood glucose, and lipid profile where its interpretability is still evident. The novel hybrid method is customizable and can be used for other disease types and/or geographic regions.

The ML model (e.g., ML model 502), also referred to as the P-CARDIAC model, and as described herein was generated to output risk prediction for recurrent CVD events among persons of a specific geographic regions (Chinese) with established CVD. Compared to previous methodologies (e.g., TRS-2° P and SMART2), ML model (e.g., ML model 502 or the P-CARDIAC model) was able to identify unique patterns of patients with established CVD with good performance. With the advantage of an ML approach the model can be calibrated periodically to account for any changes in clinical practice. The consideration of treatment effects of various drug use can also guide improved and individualized secondary prevention. For these reasons, computing applications using ML model (e.g., ML model 502 or the P-CARDIAC model) can have clinical application in a variety of settings, including primary care where real-world data will provide guidance for early intervention of lifestyle changes and potentially promote medication adherence to prevent recurrent CVD events, thus reducing the related healthcare burden.

### Additional Aspects of the Disclosure

The following aspects of the disclosure are exemplary only and not intended to limit the scope of the disclosure.

Aspect 1. A machine learning (ML)-based system for predicting cardiovascular disease, the ML-based system comprising: an ML model stored on a computer memory, the ML model trained with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training data subset comprises a remaining subset of cardiovascular risk factors; a set of computing instructions stored on the computer memory and configured to access the ML model; a processor communicatively coupled to the computer memory, and the processor configured to access the set of computing instructions and the ML model, wherein the computing instructions, when executed by the processor, cause the processor to: input user-specific cardiovascular data of a user into the ML model, wherein the user is a member of a geographic region, wherein the user-specific cardiovascular data of the user as input into the ML model is data of the user corresponding to the preselected subset of cardiovascular risk factors and the remaining subset of cardiovascular risk factors, and wherein the ML model outputs a user-specific cardiovascular prediction of the user, the user-specific cardiovascular prediction comprising a cardiovascular risk score of the user; displaying, by a graphical user interface (GUI), the user-specific cardiovascular prediction.

Aspect 2. The ML-based system of aspect 1, wherein the ML model is a Cox proportional hazards model.

Aspect 3. The ML-based system of aspect 2, wherein the computing instructions are further configured, when executed by the processor, to implement or apply a gradient boosting algorithm to the second training data subset of the remaining subset of cardiovascular risk factors to enhance the Cox proportional hazards model.

Aspect 4. The ML-based system of claim of any one of aspects 1-3, wherein each of the plurality of cardiovascular risk factors is specific to a population of the geographic region.

Aspect 5. The ML-based system of aspect 4, wherein the geographic region defining the plurality of cardiovascular risk factors on which the ML model is trained comprises a plurality subregions or cohorts comprising individuals located within each respective subregion or cohort.

Aspect 6. The ML-based system of any one of aspects 1-5, wherein the preselected subset of cardiovascular risk factors comprises risk factors selected from one or more risk categories defining indications of cardiovascular health.

Aspect 7. The ML-based system of aspect 6, wherein the one or more risk categories comprise demographic factors, family history of disease, healthcare utilization, clinical laboratory testing, medication history, disease history, and drug use.

Aspect 8. The ML-based system of any one of aspects 1-6, wherein the preselected subset of cardiovascular risk factors have a linear relationship with the ML model, and wherein the remaining subset of cardiovascular risk factors have a non-linear relationship with the ML model.

Aspect 9. The ML-based system of aspect 8, wherein the preselected subset of cardiovascular risk factors comprises one or more of values related to: age, sex, family history of diabetes, accident and emergency visits per year, aspartate transaminase, alanine aminotransferase, low-density lipoprotein cholesterol, neutrophil, statins, myocardial infarction, angina, revascularization, atrial fibrillation, hypertension, and/or user history of diabetes.

Aspect 10. The ML-based system of any one of aspects 1-9, wherein at least a portion of the preselected subset of cardiovascular risk factors comprises imputed data generated to replace missing values, and wherein the remaining subset of cardiovascular risk factors are not imputed.

Aspect 11. The ML-based system of aspect 4, wherein the ML model is further trained with data defining one or more threshold risks, where each threshold risk defines a magnitude of a clinical health benefit to a user of the geographic region.

Aspect 12. The ML-based system of any one of aspects 1-11, wherein a C-statistic for the ML model has a value of at least 0.69.

Aspect 13. The ML-based system of any one of aspects 1-12, wherein the user-specific cardiovascular prediction is a cardiovascular disease (CVD) risk prediction for the user in a 10-year timeframe.

Aspect 14. The ML-based system of any one of aspects 1-13, wherein the ML model is further trained with data of one or more drug classes identified for reducing cardiovascular disease (CVD), and wherein the user-specific cardiovascular data of the user as input into the ML model further comprises a selection of one or more of the drug classes, and wherein the user-specific cardiovascular prediction of the user comprises a CVD risk prediction that predicts the user's cardiovascular after using the one or more of the drug classes as selected.

Aspect 15. The ML-based system of any one of aspects 1-14, wherein the GUI is configured to receive the user-specific cardiovascular data of the user, and wherein the GUI is further configured to provide the user-specific cardiovascular data as input to the ML model.

Aspect 16. The ML-based system of aspect 15, wherein the GUI provides graphical fields or selections for selecting one or more types of drug classes for selection or generation of a user-specific plan to address the user's cardiovascular health.

Aspect 17. The ML-based system of any one of aspects 1-16, wherein the user-specific cardiovascular prediction comprises a user-specific medical prescription predicted to reduce the user's cardiovascular disease (CVD) risk.

Aspect 18. The ML-based system of any one of aspects 1-17, wherein the user-specific cardiovascular prediction causes generation of a user-specific activity predicted to reduce the user's cardiovascular disease (CVD) risk.

Aspect 19. A machine learning (ML)-based method for predicting cardiovascular disease, the ML-based method comprising: training, by one or more processors, an ML model with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training subset comprises a remaining subset of cardiovascular risk factors; inputting, by the one or more processors, user-specific cardiovascular data of a user into the ML model, wherein the user is a member of a geographic region, and wherein the user-specific cardiovascular data of the user as input into the ML model is data of the user corresponding to the preselected subset of cardiovascular risk factors and the remaining subset of cardiovascular risk factors; outputting, by the one or more processors accessing the ML model, a user-specific cardiovascular prediction of the user, the user-specific cardiovascular prediction comprising a cardiovascular risk score of the user; and displaying, by the one or more processors, the user-specific cardiovascular prediction on a graphical user interface (GUI).

Aspect 20. The ML-based method of aspect 19, wherein the ML model is a Cox proportional hazards model.

Aspect 21. The ML-based method of aspect 20, further comprising implementing or applying a gradient boosting algorithm to the second training data subset of the remaining subset of cardiovascular risk factors to enhance the Cox proportional hazards model.

Aspect 22. The ML-based method of any one of aspects 19-21, wherein each of the plurality of cardiovascular risk factors is specific to a population of a geographic region

Aspect 23. The ML-based method of any one of aspects 19-22, wherein the geographic region defining the plurality of cardiovascular risk factors on which the ML model is trained comprises a plurality subregions or cohorts comprising individuals located within each respective subregion or cohort.

Aspect 24. The ML-based method of any one of aspects 19-23, wherein the preselected subset of cardiovascular risk factors comprises risk factors selected from one or more risk categories defining indications of cardiovascular health.

Aspect 25. The ML-based method of aspect 24, wherein the one or more risk categories comprise demographic factors, family history of disease, healthcare utilization, clinical laboratory testing, medication history, disease history, and drug use.

Aspect 26. The ML-based method of any one of aspects 19-25, wherein the preselected subset of cardiovascular risk factors have a linear relationship with the ML model, and wherein the remaining subset of cardiovascular risk factors have a non-linear relationship with the ML model.

Aspect 27. The ML-based method of aspect 26, wherein the preselected subset of cardiovascular risk factors comprises one or more of values related to: age, sex, family history of diabetes, accident and emergency visits per year, aspartate transaminase, alanine aminotransferase, low-density lipoprotein cholesterol, neutrophil, statins, myocardial infarction, angina, revascularization, atrial fibrillation, hypertension, and/or user history of diabetes.

Aspect 28. The ML-based method of any one of aspects 19-27, wherein at least a portion of the preselected subset of cardiovascular risk factors comprises imputed data generated to replace missing values, and wherein the remaining subset of cardiovascular risk factors are not imputed.

Aspect 29. The ML-based method of any one of aspects 19-28, wherein the ML model is further trained with data defining one or more threshold risks, where each threshold risk defines a magnitude of a clinical health benefit to a user of the geographic region.

Aspect 30. The ML-based method of any one of aspects 19-29, wherein a C-statistic for the ML model has a value of at least 0.69.

Aspect 31. The ML-based method of any one of aspects 19-30, wherein the user-specific cardiovascular prediction is a cardiovascular disease (CVD) risk prediction for the user in a 10-year timeframe.

Aspect 32. The ML-based method of any one of aspects 19-31, wherein the ML model is further trained with data of one or more drug classes identified for reducing cardiovascular disease (CVD), and wherein the user-specific cardiovascular data of the user as input into the ML model further comprises a selection of one or more of the drug classes, and wherein the user-specific cardiovascular prediction of the user comprises a CVD risk prediction that predicts the user's cardiovascular after using the one or more of the drug classes as selected.

Aspect 33. The ML-based method of any one of aspects 19-32, wherein the GUI is configured to receive the user-specific cardiovascular data of the user, and wherein the GUI is further configured to provide the user-specific cardiovascular data as input to the ML model.

Aspect 34. The ML-based method of aspect 33, wherein the GUI provides graphical fields or selections for selecting one or more types of drug classes for selection or generation of a user-specific plan to address the user's cardiovascular health.

Aspect 35. The ML-based method of any one of aspects 19-34, wherein the user-specific cardiovascular prediction comprises a user-specific medical prescription predicted to reduce the user's cardiovascular disease (CVD) risk.

Aspect 36. The ML-based method of any one of aspects 19-35, wherein the user-specific cardiovascular prediction causes generation of a user-specific activity predicted to reduce the user's cardiovascular disease (CVD) risk.

Aspect 37. A tangible, non-transitory computer-readable medium storing computing instructions for predicting cardiovascular disease, that when executed by one or more processors cause the one or more processors to: train an ML model with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training subset comprises a remaining subset of cardiovascular risk factors, input user-specific cardiovascular data of a user into an ML model stored on a computer memory, wherein the user is a member of a geographic region, and wherein the user-specific cardiovascular data of the user as input into the ML model is data of the user corresponding to the preselected subset of cardiovascular risk factors and the remaining subset of cardiovascular risk factors, output, by the ML model, a user-specific cardiovascular prediction of the user, the user-specific cardiovascular prediction comprising a cardiovascular risk score of the user; and display, by a graphical user interface (GUI), the user-specific cardiovascular prediction.

Aspect 38. The tangible, non-transitory computer-readable medium of aspect 37, wherein the ML model is a Cox proportional hazards model.

Aspect 39. The tangible, non-transitory computer-readable medium of aspect 38, wherein the computing instructions are further configured, when executed by the processor, to implement or apply a gradient boosting algorithm to the second training data subset of the remaining subset of cardiovascular risk factors to enhance the Cox proportional hazards model.

Aspect 40. The tangible, non-transitory computer-readable medium of any one of aspects 37-39, wherein each of the plurality of cardiovascular risk factors is specific to a population of a geographic region.

Aspect 41. The tangible, non-transitory computer-readable medium of any one of aspects 37-40, wherein the geographic region defining the plurality of cardiovascular risk factors on which the ML model is trained comprises a plurality subregions or cohorts comprising individuals located within each respective subregion or cohort.

Aspect 42. The tangible, non-transitory computer-readable medium of any one of aspects 37-41, wherein the preselected subset of cardiovascular risk factors comprises risk factors selected from one or more risk categories defining indications of cardiovascular health.

Aspect 43. The tangible, non-transitory computer-readable medium of aspect 42, wherein the one or more risk categories comprise demographic factors, family history of disease, healthcare utilization, clinical laboratory testing, medication history, disease history, and drug use.

Aspect 44. The tangible, non-transitory computer-readable medium of any one of aspects 37-43, wherein the preselected subset of cardiovascular risk factors have a linear relationship with the ML model, and wherein the remaining subset of cardiovascular risk factors have a non-linear relationship with the ML model.

Aspect 45. The tangible, non-transitory computer-readable medium of aspect 44, wherein the preselected subset of cardiovascular risk factors comprises one or more of values related to: age, sex, family history of diabetes, accident and emergency visits per year, aspartate transaminase, alanine aminotransferase, low-density lipoprotein cholesterol, neutrophil, statins, myocardial infarction, angina, revascularization, atrial fibrillation, hypertension, and/or user history of diabetes.

Aspect 46. The tangible, non-transitory computer-readable medium of any one of aspects 37-45, wherein at least a portion of the preselected subset of cardiovascular risk factors comprises imputed data generated to replace missing values, and wherein the remaining subset of cardiovascular risk factors are not imputed.

Aspect 47. The tangible, non-transitory computer-readable medium of any one of aspects 37-46, wherein the ML model is further trained with data defining one or more threshold risks, where each threshold risk defines a magnitude of a clinical health benefit to a user of the geographic region.

Aspect 48. The tangible, non-transitory computer-readable medium of any one of aspects 37-47, wherein a C-statistic for the ML model has a value of at least 0.69.

Aspect 49. The tangible, non-transitory computer-readable medium of any one of aspects 37-48, wherein the user-specific cardiovascular prediction is a cardiovascular disease (CVD) risk prediction for the user in a 10-year timeframe.

Aspect 50. The tangible, non-transitory computer-readable medium of any one of aspects 37-49, wherein the ML model is further trained with data of one or more drug classes identified for reducing cardiovascular disease (CVD), and wherein the user-specific cardiovascular data of the user as input into the ML model further comprises a selection of one or more of the drug classes, and wherein the user-specific cardiovascular prediction of the user comprises a CVD risk prediction that predicts the user's cardiovascular after using the one or more of the drug classes as selected.

Aspect 51. The tangible, non-transitory computer-readable medium of any one of aspects 37-50, wherein the GUI is configured to receive the user-specific cardiovascular data of the user, and wherein the GUI is further configured to provide the user-specific cardiovascular data as input to the ML model.

Aspect 52. The tangible, non-transitory computer-readable medium of aspect 51, wherein the GUI provides graphical fields or selections for selecting one or more types of drug classes for selection or generation of a user-specific plan to address the user's cardiovascular health.

Aspect 53. The tangible, non-transitory computer-readable medium of any one of aspects 37-52, wherein the user-specific cardiovascular prediction comprises a user-specific medical prescription predicted to reduce the user's cardiovascular disease (CVD) risk.

Aspect 54. The tangible, non-transitory computer-readable medium of any one of aspects 37-53, wherein the user-specific cardiovascular prediction causes generation of a user-specific activity predicted to reduce the user's cardiovascular disease (CVD) risk.

Aspect 55. A machine learning (ML)-based method for predicting disease, the ML-based method comprising: training, by one or more processors, an ML model with data of a plurality of disease risk factors specific to a population of a given geographic region, the plurality of disease risk factors subdivided into a first training data subset and a second training data subset prior to training the ML model, wherein the first training data subset comprises a preselected subset of disease risk factors, and wherein the second training subset comprises a remaining subset of disease risk factors, inputting, by the one or more processors, user-specific health data of a user into the ML model, wherein the user is a member of the geographic region, and wherein the user-specific health data of the user as input into the ML model is data of the user corresponding to the preselected subset of disease risk factors and the remaining subset of disease risk factors, outputting, by the one or more processors accessing the ML model, a user-specific disease prediction of the user, the user-specific disease prediction comprising a disease risk score of the user; and displaying, by the one or more processors, the user-specific disease prediction on a graphical user interface (GUI).

### Additional Considerations

Although the disclosure herein sets forth a detailed description of numerous different embodiments, it should be understood that the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method may be performed by one or more processors or processor-implemented hardware modules. The performance of certain of the operations may be distributed among the one or more processors, not only residing within a single machine, but deployed across a number of machines. In some example embodiments, the processor or processors may be located in a single location, while in other embodiments the processors may be distributed across a number of locations.

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other embodiments, the one or more processors or processor-implemented modules may be distributed across a number of geographic locations.

This detailed description is to be construed as exemplary only and does not describe every possible embodiment, as describing every possible embodiment would be impractical, if not impossible. A person of ordinary skill in the art may implement numerous alternate embodiments, using either current technology or technology developed after the filing date of this application.

Those of ordinary skill in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

The patent claims at the end of this patent application are not intended to be construed under 35 U.S.C. § 112(f) unless traditional means-plus-function language is expressly recited, such as “means for” or “step for” language being explicitly recited in the claim(s). The systems and methods described herein are directed to an improvement to computer functionality and improve the functioning of conventional computers.

1. A machine learning (ML)-based system for predicting cardiovascular disease, the ML-based system comprising:
an ML model stored on a computer memory, the ML model trained with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training data subset comprises a remaining subset of cardiovascular risk factors;
a set of computing instructions stored on the computer memory and configured to access the ML model;
a processor communicatively coupled to the computer memory, and the processor configured to access the set of computing instructions and the ML model, wherein the computing instructions, when executed by the processor, cause the processor to:
input user-specific cardiovascular data of a user into the ML model, wherein the user is a member of a geographic region, wherein the user-specific cardiovascular data of the user as input into the ML model is data of the user corresponding to the preselected subset of cardiovascular risk factors and the remaining subset of cardiovascular risk factors, and wherein the ML model outputs a user-specific cardiovascular prediction of the user, the user-specific cardiovascular prediction comprising a cardiovascular risk score of the user;
displaying, by a graphical user interface (GUI), the user-specific cardiovascular prediction.

2. The ML-based system of claim 1, wherein the ML model is a Cox proportional hazards model, wherein the computing instructions are further configured, when executed by the processor, to implement or apply a gradient boosting algorithm to the second training data subset of the remaining subset of cardiovascular risk factors to enhance the Cox proportional hazards model.

3. (canceled)

4. The ML-based system of claim 1, wherein each of the plurality of cardiovascular risk factors is specific to a population of the geographic region, and wherein the geographic region defining the plurality of cardiovascular risk factors on which the ML model is trained comprises a plurality subregions or cohorts comprising individuals located within each respective subregion or cohort.

5. (canceled)

6. The ML-based system of claim 1, wherein the preselected subset of cardiovascular risk factors comprises risk factors selected from one or more risk categories defining indications of cardiovascular health, and wherein the one or more risk categories comprise demographic factors, family history of disease, healthcare utilization, clinical laboratory testing, medication history, disease history, and drug use.

7. (canceled)

8. The ML-based system of claim 1, wherein the preselected subset of cardiovascular risk factors have a linear relationship with the ML model, and wherein the remaining subset of cardiovascular risk factors have a non-linear relationship with the ML model, and wherein the preselected subset of cardiovascular risk factors comprises one or more of values related to: age, sex, family history of diabetes, accident and emergency visits per year, aspartate transaminase, alanine aminotransferase, low-density lipoprotein cholesterol, neutrophil, statins, myocardial infarction, angina, revascularization, atrial fibrillation, hypertension, and/or user history of diabetes.

9. (canceled)

10. The ML-based system of claim 1, wherein at least a portion of the preselected subset of cardiovascular risk factors comprises imputed data generated to replace missing values, and wherein the remaining subset of cardiovascular risk factors are not imputed, and wherein the ML model is further trained with data defining one or more threshold risks, where each threshold risk defines a magnitude of a clinical health benefit to a user of the geographic region.

11. (canceled)

12. The ML-based system of claim 1, wherein a C-statistic for the ML model has a value of at least 0.69.

13. The ML-based system of claim 1, wherein the user-specific cardiovascular prediction is a cardiovascular disease (CVD) risk prediction for the user in a 10-year timeframe.

14. The ML-based system of claim 1, wherein the ML model is further trained with data of one or more drug classes identified for reducing cardiovascular disease (CVD), and wherein the user-specific cardiovascular data of the user as input into the ML model further comprises a selection of one or more of the drug classes, and wherein the user-specific cardiovascular prediction of the user comprises a CVD risk prediction that predicts the user's cardiovascular after using the one or more of the drug classes as selected.

15. The ML-based system of claim 1, wherein the GUI is configured to receive the user-specific cardiovascular data of the user, and wherein the GUI is further configured to provide the user-specific cardiovascular data as input to the ML model, wherein the GUI provides graphical fields or selections for selecting one or more types of drug classes for selection or generation of a user-specific plan to address the user's cardiovascular health.

16. (canceled)

17. The ML-based system of claim 1, wherein the user-specific cardiovascular prediction comprises at least one of: a user-specific medical prescription predicted to reduce the user's cardiovascular disease (CVD) risk or causes generation of a user-specific activity predicted to reduce the user's cardiovascular disease (CVD) risk.

18. (canceled)

19. A machine learning (ML)-based method for predicting cardiovascular disease, the ML-based method comprising:
training, by one or more processors, an ML model with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training subset comprises a remaining subset of cardiovascular risk factors;
inputting, by the one or more processors, user-specific cardiovascular data of a user into the ML model, wherein the user is a member of a geographic region, and wherein the user-specific cardiovascular data of the user as input into the ML model is data of the user corresponding to the preselected subset of cardiovascular risk factors and the remaining subset of cardiovascular risk factors;
outputting, by the one or more processors accessing the ML model, a user-specific cardiovascular prediction of the user, the user-specific cardiovascular prediction comprising a cardiovascular risk score of the user; and
displaying, by the one or more processors, the user-specific cardiovascular prediction on a graphical user interface (GUI).

20. The ML-based method of claim 19, wherein the ML model is a Cox proportional hazards model, and wherein the ML-based method further comprises implementing or applying a gradient boosting algorithm to the second training data subset of the remaining subset of cardiovascular risk factors to enhance the Cox proportional hazards model.

21. (canceled)

22. The ML-based method of claim 19, wherein each of the plurality of cardiovascular risk factors is specific to a population of a geographic region.

23. The ML-based method of claim 19, wherein the geographic region defining the plurality of cardiovascular risk factors on which the ML model is trained comprises a plurality subregions or cohorts comprising individuals located within each respective subregion or cohort.

24. The ML-based method of claim 19, wherein the preselected subset of cardiovascular risk factors comprises risk factors selected from one or more risk categories defining indications of cardiovascular health, and wherein the one or more risk categories comprise demographic factors, family history of disease, healthcare utilization, clinical laboratory testing, medication history, disease history, and drug use.

25. (canceled)

26. The ML-based method of claim 19, wherein the preselected subset of cardiovascular risk factors have a linear relationship with the ML model, and wherein the remaining subset of cardiovascular risk factors have a non-linear relationship with the ML model, and wherein the preselected subset of cardiovascular risk factors comprises one or more of values related to: age, sex, family history of diabetes, accident and emergency visits Per year, aspartate transaminase, alanine aminotransferase, low-density lipoprotein cholesterol, neutrophil, statins, myocardial infarction, angina, revascularization, atrial fibrillation, hypertension, and/or user history of diabetes.

27. (canceled)

28. The ML-based method of claim 19, wherein at least a portion of the preselected subset of cardiovascular risk factors comprises imputed data generated to replace missing values, and wherein the remaining subset of cardiovascular risk factors are not imputed.

29. The ML-based method of claim 19, wherein the ML model is further trained with data defining one or more threshold risks, where each threshold risk defines a magnitude of a clinical health benefit to a user of the geographic region.

30. The ML-based method of claim 19, wherein a C-statistic for the ML model has a value of at least 0.69.

31. The ML-based method of claim 19, wherein the user-specific cardiovascular prediction is a cardiovascular disease (CVD) risk prediction for the user in a 10-year timeframe.

32. The ML-based method of claim 19, wherein the ML model is further trained with data of one or more drug classes identified for reducing cardiovascular disease (CVD), and wherein the user-specific cardiovascular data of the user as input into the ML model further comprises a selection of one or more of the drug classes, and wherein the user-specific cardiovascular prediction of the user comprises a CVD risk prediction that predicts the user's cardiovascular after using the one or more of the drug classes as selected.

33. The ML-based method of claim 19, wherein the GUI is configured to receive the user-specific cardiovascular data of the user, and wherein the GUI is further configured to provide the user-specific cardiovascular data as input to the ML model, and wherein the GUI provides graphical fields or selections for selectinq one or more types of drug classes for selection or generation of a user-specific plan to address the user's cardiovascular health.

34. (canceled)

35. The ML-based method of claim 19, wherein the user-specific cardiovascular prediction comprises at least one of: a user-specific medical prescription predicted to reduce the user's cardiovascular disease (CVD) risk, or causes generation of a user-specific activity predicted to reduce the user's cardiovascular disease (CVD) risk.

36. (canceled)

37. A tangible, non-transitory computer-readable medium storing computing instructions for predicting cardiovascular disease, that when executed by one or more processors cause the one or more processors to:
train an ML model with data of a plurality of cardiovascular risk factors, the plurality of cardiovascular risk factors subdivided into a first training data subset and a second training data subset prior to training the ML model, wherein the first training data subset comprises a preselected subset of cardiovascular risk factors, and wherein the second training subset comprises a remaining subset of cardiovascular risk factors,
input user-specific cardiovascular data of a user into an ML model stored on a computer memory, wherein the user is a member of a geographic region, and wherein the user-specific cardiovascular data of the user as input into the ML model is data of the user corresponding to the preselected subset of cardiovascular risk factors and the remaining subset of cardiovascular risk factors,
output, by the ML model, a user-specific cardiovascular prediction of the user, the user-specific cardiovascular prediction comprising a cardiovascular risk score of the user; and
display, by a graphical user interface (GUI), the user-specific cardiovascular prediction.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. A machine learning (ML)-based method for predicting disease, the ML-based method comprising:
training, by one or more processors, an ML model with data of a plurality of disease risk factors specific to a population of a given geographic region, the plurality of disease risk factors subdivided into a first training data subset and a second training data subset prior to training the ML model, wherein the first training data subset comprises a preselected subset of disease risk factors, and wherein the second training subset comprises a remaining subset of disease risk factors,
inputting, by the one or more processors, user-specific health data of a user into the ML model, wherein the user is a member of the geographic region, and wherein the user-specific health data of the user as input into the ML model is data of the user corresponding to the preselected subset of disease risk factors and the remaining subset of disease risk factors,
outputting, by the one or more processors accessing the ML model, a user-specific disease prediction of the user, the user-specific disease prediction comprising a disease risk score of the user; and
displaying, by the one or more processors, the user-specific disease prediction on a graphical user interface (GUI).
